(19) United States
(12) Patent Application Publication
(10) Pub. No.: US 2023/0410957 A1
(43) Pub. Date: Dec. 21, 2023

(54) METHODS AND SYSTEMS FOR CONDUCTING PREGNANCY-RELATED CLINICAL TRIALS

(71) Applicant: Mirvie, Inc., South San Francisco, CA (US)

(72) Inventors: Maneesh Jain, San Francisco, CA (US); Farida Peters-Abbadi, Marblehead, MA (US); Manfred Lee, Belmont, CA (US); Eugeni Namsaraev, Santa Clara, CA (US)

(21) Appl. No.: 18/230,328

(22) Filed: Aug. 4, 2023

Related U.S. Application Data
(63) Continuation of application No. PCT/US2022/015754, filed on Feb. 9, 2022.
(60) Provisional application No. 63/148,032, filed on Feb. 10, 2021.

(51) Int. Cl. G16H 10/20; G06Q 30/0251

(52) U.S. Cl. CPC G16H 10/20; G06Q 30/0271

(57) ABSTRACT

The present disclosure provides methods and systems for directing pregnancy-related clinical trials. A method for directing a clinical trial may comprise evaluating a subject for participation in a clinical trial for assessing pregnancy-related states of subjects or fetuses thereof, selecting the subject for the clinical trial, based at least in part on the evaluating, and directing collection of a biological sample of the subject at a location remote with respect to a health care facility.

### CROSS-REFERENCE

This application is a continuation of International Application No. PCT/US2022/015754, filed Feb. 9, 2022, which claims the benefit of U.S. Provisional Application No. 63/148,032, filed Feb. 10, 2021, each of which is incorporated by reference herein in its entirety.

### BACKGROUND

Pregnancy-related complications such as pre-term birth are a leading cause of neonatal death and of complications later in life. Further, such pregnancy-related complications can cause negative health effects on maternal health. Clinical trials may be directed to identifying and monitoring pregnancy-related states of subjects or their fetuses using non-invasive and cost-effective approaches, toward improving maternal and fetal health.

### SUMMARY

Current pregnancy-related clinical trials may be inaccessible and incomplete. For cases in which pregnancies progress without pregnancy-related complications, limited methods of pregnancy monitoring may be available for a pregnancy subject, such as molecular tests, ultrasound imaging, and estimation of gestational age and/or due date using the last menstrual period. However, such monitoring methods may be complex, expensive, and unreliable. For example, molecular tests cannot predict gestational age, ultrasound imaging is expensive and best performed during the first trimester of pregnancy, and estimation of gestational age and/or due date using the last menstrual period can be unreliable. Further, for cases in which pregnancies progress with pregnancy-related complications such as risk of spontaneous pre-term delivery, the clinical utility of molecular tests, ultrasound imaging, and demographic factors may be limited. For example, molecular tests may have a limited BMI (body mass index) range, a limited gestational age and/or due date range (about 2 weeks), and a low positive predictive value (PPV); ultrasound imaging may be expensive and have low PPV and specificity; and the use of demographic factors to predict risk of pregnancy-related complications may be unreliable. Therefore, there exists an urgent clinical need for accurate pregnancy-related clinical trials for detection and monitoring of pregnancy-related states (e.g., estimation of gestational age, due date, and/or onset of labor, and prediction of pregnancy-related complications such as pre-term birth) toward clinically actionable outcomes.

The present disclosure provides methods and systems for directing a clinical trial for assessing pregnancy-related states of subjects or fetuses thereof. For example, the subjects may include subjects with one or more pregnancy-related states and subjects without pregnancy-related states. A method for directing a clinical trial may comprise evaluating a subject for participation in a clinical trial for assessing pregnancy-related states of subjects or fetuses thereof, selecting the subject for the clinical trial, based at least in part on the evaluating, and directing collection of a biological sample of the subject at a location remote with respect to a health care facility. A method for directing a clinical trial may comprise evaluating a subject for participation in a clinical trial for assessing pregnancy-related states of fetuses of subjects, and selecting the subject for the clinical trial, based at least in part on the evaluating, wherein the clinical trial comprises collection of data in absence of the subject visiting a health care facility. A method for directing a clinical trial may comprise evaluating a subject for participation in a clinical trial for assessing pregnancy-related states of subjects or fetuses thereof, wherein the subjects are in a first trimester or a second trimester of pregnancy, and selecting the subject for the clinical trial, based at least in part on the evaluating, wherein the clinical trial comprises collection of data in absence of the subject visiting a health care facility.

Pregnancy-related states may include, for example, pre-term birth, full-term birth, gestational age, due date (e.g., due date for an unborn baby or fetus of a subject), onset of labor, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications, hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), fertility-related conditions, and fetal development stages or states (e.g., normal fetal organ function or development, and abnormal fetal organ function or development). For example, post-partum complications may include cardiovascular diseases (e.g., peripartum cardiomyopathy (PPCM), also known as postpartum cardiomyopathy), depression and anxiety (e.g., post-partum depression), post GDM (e.g., post-partum glucose tolerance to diabetes progression for a mother or infant), post-partum complications of preeclampsia (e.g., post-partum preeclampsia), excessive bleeding after giving birth (e.g., hemorrhage, placenta accreta), pulmonary embolism, cardiomyopathy, diabetes, anemia, and hypertensive disorders.

In an aspect, the present disclosure provides a method for directing a clinical trial, comprising: (a) evaluating a subject for participation in the clinical trial, wherein the clinical trial is for assessing pregnancy-related states of subjects or fetuses thereof; (b) selecting the subject for the clinical trial, based at least in part on the evaluating in (a); and (c) directing collection of a biological sample of the subject at a location remote with respect to a health care facility.

In some embodiments, the subject is pregnant or is suspected of being pregnant. In some embodiments, the pregnancy-related states of the subjects or the fetuses thereof comprise prenatal pregnancy-related states of the subjects or the fetuses thereof. In some embodiments, the pregnancy-related states of the subjects or the fetuses thereof comprise postnatal pregnancy-related states of the subjects or the fetuses thereof.

In some embodiments, the pregnancy-related states are selected from the group consisting of pre-term birth, full-term birth, gestational age, due date, onset of labor, a pregnancy-related hypertensive disorder, preeclampsia, eclampsia, gestational diabetes, a congenital disorder of a fetus of the subject, ectopic pregnancy, spontaneous abortion, stillbirth, a post-partum complication, hyperemesis gravidarum, hemorrhage or excessive bleeding during delivery, premature rupture of membrane, premature rupture of membrane in pre-term birth, placenta previa, intrauterine fetal growth restriction, macrosomia, a neonatal condition, a fertility-related condition, and a fetal development stage or state. In some embodiments, the pregnancy-related states comprise the pre-term birth. In some embodiments, the pregnancy-related states comprise the pre-eclampsia. In some embodiments, the pregnancy-related states comprise the due date.

In some embodiments, the method further comprises recruiting the subject for participation in the clinical trial. In some embodiments, the recruiting comprises use of a digital marketing campaign. In some embodiments, the recruiting comprises displaying advertisements to the subject through a computer network. In some embodiments, the advertisements are displayed through mobile device of the subject. In some embodiments, the mobile device comprises a smartphone, a laptop, a tablet computer, a smartwatch, or a combination thereof. In some embodiments, the advertisements are displayed or viewed through a social media channel, social networking, pregnancy education, pregnancy tracker, menstruation tracker, ovulation tracker, or fertility tracker platform. In some embodiments, recruiting the subject comprises, responsive to the subject responding to the displayed advertisement, directing the subject to visit or view one or more web pages of a user experience (UX) journey. In some embodiments, the subject responds to the displayed advertisement by clicking a hyperlink of the displayed advertisement. In some embodiments, recruiting the subject comprises displaying a contact form to the subject for completing, and receiving the completed contact form comprising personal contact information from the subject.

In some embodiments, the method further comprises displaying an eligibility questionnaire or a set of eligibility criteria to the subject via the one or more web pages. In some embodiments, the method further comprises collecting response data from the subject using the eligibility questionnaire, a self-reported family history survey, a demographic survey, a medical condition survey, a customer satisfaction survey, or a combination thereof. In some embodiments, evaluating the subject for participation in the clinical trial comprises processing the response data collected from the subject. In some embodiments, processing the response data collected from the subject comprises determining a gestational age of the fetus of the subject and/or a time window for the collection of the biological sample of the subject. In some embodiments, evaluating the subject for participation in the clinical trial comprises applying an inclusion criterion or an exclusion criterion of the clinical trial. In some embodiments, the inclusion criterion or the exclusion criterion is related to age, race, ethnicity, body mass index, gestational age, or location of the subject or a fetus of the subject.

In some embodiments, the method further comprises selecting the subject for the clinical trial when the subject satisfies the inclusion criterion. In some embodiments, the method further comprises obtaining informed consent from the subject. the informed consent is obtained virtually using electronic signatures. In some embodiments, the informed consent is obtained for research use of the biological sample of the subject and data resulting therefrom. In some embodiments, the informed consent is obtained for access to at least a portion of medical records of the subject. In some embodiments, the informed consent comprises authorization for a health care provider to release the medical records of the subject. In some embodiments, the method further comprises excluding the subject for the clinical trial when the subject satisfies the exclusion criterion.

In some embodiments, the biological sample is selected from the group consisting of a blood sample, a urine sample, a stool sample, a saliva sample, a vaginal sample, a cervical sample, and a swab sample. In some embodiments, the biological sample comprises the blood sample. In some embodiments, (c) comprises shipping a biological sample collection kit to the location, and receiving a return shipment of the collected biological sample from the location. In some embodiments, the blood sample is obtained from the subject by a phlebotomy service. In some embodiments, the phlebotomy service is a mobile phlebotomy service. In some embodiments, (c) comprises shipping a biological sample collection kit to a phlebotomist, and receiving a return shipment of the collected biological sample from the phlebotomist. In some embodiments, the location is a residence, a workplace, or another location of choice of the subject.

In some embodiments, the clinical trial is an interventional clinical trial. In some embodiments, the interventional clinical trial is directed at least in part through a telemedicine consultation with a health care provider of the subject. In some embodiments, the clinical trial is an observational clinical trial. In some embodiments, the clinical trial is a longitudinal clinical trial. In some embodiments, the clinical trial is an Institutional Review Board (IRB) approved clinical trial.

In another aspect, the present disclosure provides a method for directing a clinical trial, comprising: (a) evaluating a subject for participation in the clinical trial, wherein the clinical trial is for assessing pregnancy-related states of fetuses of subjects; and (b) selecting the subject for the clinical trial, based at least in part on the evaluating in (a), wherein the clinical trial comprises collection of data in absence of the subject visiting a health care facility.

In some embodiments, the subject is pregnant or is suspected of being pregnant. In some embodiments, the pregnancy-related states of the fetuses of the subjects comprise prenatal pregnancy-related states of the fetuses of the subjects. In some embodiments, the pregnancy-related states of the fetuses of the subjects comprise postnatal pregnancy-related states of the fetuses of the subjects.

In some embodiments, the pregnancy-related states are selected from the group consisting of pre-term birth, full-term birth, gestational age, due date, onset of labor, a congenital disorder of the fetus of the subject, ectopic pregnancy, spontaneous abortion, stillbirth, intrauterine fetal growth restriction, macrosomia, a neonatal condition, a fertility-related condition, and a fetal development stage or state. In some embodiments, the pregnancy-related states comprise the pre-term birth. In some embodiments, the pregnancy-related states comprise the due date.

In some embodiments, the method further comprises recruiting the subject for participation in the clinical trial. In some embodiments, the recruiting comprises use of a digital marketing campaign. In some embodiments, the recruiting comprises displaying advertisements to the subject through a computer network. In some embodiments, the advertisements are displayed through mobile device of the subject. In some embodiments, the mobile device comprises a smartphone, a laptop, a tablet computer, a smartwatch, or a combination thereof. In some embodiments, the advertisements are displayed or viewed through a social media channel, social networking, pregnancy education, pregnancy tracker, menstruation tracker, ovulation tracker, or fertility tracker platform. In some embodiments, recruiting the subject comprises, responsive to the subject responding to the displayed advertisement, directing the subject to visit or view one or more web pages of a user experience (UX) journey. In some embodiments, the subject responds to the displayed advertisement by clicking a hyperlink of the displayed advertisement. In some embodiments, recruiting the subject comprises displaying a contact form to the subject for completing, and receiving the completed contact form comprising personal contact information from the subject.

In some embodiments, the method further comprises displaying an eligibility questionnaire or a set of eligibility criteria to the subject via the one or more web pages. In some embodiments, the method further comprises collecting response data from the subject using the eligibility questionnaire, a self-reported family history survey, a demographic survey, a medical condition survey, a customer satisfaction survey, or a combination thereof. In some embodiments, evaluating the subject for participation in the clinical trial comprises processing the response data collected from the subject. In some embodiments, processing the response data collected from the subject comprises determining a gestational age of the fetus of the subject and/or a time window for the collection of the biological sample of the subject. In some embodiments, evaluating the subject for participation in the clinical trial comprises applying an inclusion criterion or an exclusion criterion of the clinical trial. In some embodiments, the inclusion criterion or the exclusion criterion is related to age, race, ethnicity, body mass index, gestational age, or location of the subject or a fetus of the subject.

In some embodiments, the method further comprises selecting the subject for the clinical trial when the subject satisfies the inclusion criterion. In some embodiments, the method further comprises obtaining informed consent from the subject. the informed consent is obtained virtually using electronic signatures. In some embodiments, the informed consent is obtained for research use of the biological sample of the subject and data resulting therefrom. In some embodiments, the informed consent is obtained for access to at least a portion of medical records of the subject. In some embodiments, the informed consent comprises authorization for a health care provider to release the medical records of the subject. In some embodiments, the method further comprises excluding the subject for the clinical trial when the subject satisfies the exclusion criterion.

In some embodiments, the collection of data comprises assaying a biological sample of the subject. In some embodiments, the biological sample is selected from the group consisting of a blood sample, a urine sample, a stool sample, a saliva sample, a vaginal sample, a cervical sample, and a swab sample. In some embodiments, the biological sample comprises the blood sample. In some embodiments, the collection of data comprises shipping a biological sample collection kit to the location, and receiving a return shipment of the collected biological sample from the location. In some embodiments, the blood sample is obtained from the subject by a phlebotomy service. In some embodiments, the phlebotomy service is a mobile phlebotomy service. In some embodiments, the collection of data comprises shipping a biological sample collection kit to a phlebotomist, and receiving a return shipment of the collected biological sample from the phlebotomist. In some embodiments, the location is a residence, a workplace, or another location of choice of the subject.

In some embodiments, the clinical trial is an interventional clinical trial. In some embodiments, the interventional clinical trial is directed at least in part through a telemedicine consultation with a health care provider of the subject. In some embodiments, the clinical trial is an observational clinical trial. In some embodiments, the clinical trial is a longitudinal clinical trial. In some embodiments, the clinical trial is an Institutional Review Board (IRB) approved clinical trial.

In another aspect, the present disclosure provides a method for directing a clinical trial, comprising: (a) evaluating a subject for participation in the clinical trial, wherein the clinical trial is for assessing pregnancy-related states of subjects or fetuses thereof, wherein the subjects are in a first trimester or a second trimester of pregnancy; and (b) selecting the subject for the clinical trial, based at least in part on the evaluating in (a), wherein the clinical trial comprises collection of data in absence of the subject visiting a health care facility.

In some embodiments, the subject is pregnant or is suspected of being pregnant. In some embodiments, the pregnancy-related states of the subjects or the fetuses thereof comprise prenatal pregnancy-related states of the subjects or the fetuses thereof. In some embodiments, the pregnancy-related states of the subjects or the fetuses thereof comprise postnatal pregnancy-related states of the subjects or the fetuses thereof.

In some embodiments, the pregnancy-related states are selected from the group consisting of pre-term birth, full-term birth, gestational age, due date, onset of labor, a pregnancy-related hypertensive disorder, preeclampsia, eclampsia, gestational diabetes, a congenital disorder of a fetus of the subject, ectopic pregnancy, spontaneous abortion, stillbirth, a post-partum complication, hyperemesis gravidarum, hemorrhage or excessive bleeding during delivery, premature rupture of membrane, premature rupture of membrane in pre-term birth, placenta previa, intrauterine fetal growth restriction, macrosomia, a neonatal condition, a fertility-related condition, and a fetal development stage or state. In some embodiments, the pregnancy-related states comprise the pre-term birth. In some embodiments, the pregnancy-related states comprise the due date.

In some embodiments, the method further comprises recruiting the subject for participation in the clinical trial. In some embodiments, the recruiting comprises use of a digital marketing campaign. In some embodiments, the recruiting comprises displaying advertisements to the subject through a computer network. In some embodiments, the advertisements are displayed through mobile device of the subject. In some embodiments, the mobile device comprises a smartphone, a laptop, a tablet computer, a smartwatch, or a combination thereof. In some embodiments, the advertisements are displayed or viewed through a social media channel, social networking, pregnancy education, pregnancy tracker, menstruation tracker, ovulation tracker, or fertility tracker platform. In some embodiments, recruiting the subject comprises, responsive to the subject responding to the displayed advertisement, directing the subject to visit or view one or more web pages of a user experience (UX) journey. In some embodiments, the subject responds to the displayed advertisement by clicking a hyperlink of the displayed advertisement. In some embodiments, recruiting the subject comprises displaying a contact form to the subject for completing, and receiving the completed contact form comprising personal contact information from the subject.

In some embodiments, the method further comprises displaying an eligibility questionnaire or a set of eligibility criteria to the subject via the one or more web pages. In some embodiments, the method further comprises collecting response data from the subject using the eligibility questionnaire, a self-reported family history survey, a demographic survey, a medical condition survey, a customer satisfaction survey, or a combination thereof. In some embodiments, evaluating the subject for participation in the clinical trial comprises processing the response data collected from the subject. In some embodiments, processing the response data collected from the subject comprises determining a gestational age of the fetus of the subject and/or a time window for the collection of the biological sample of the subject. In some embodiments, evaluating the subject for participation in the clinical trial comprises applying an inclusion criterion or an exclusion criterion of the clinical trial. In some embodiments, the inclusion criterion or the exclusion criterion is related to age, race, ethnicity, body mass index, gestational age, or location of the subject or a fetus of the subject.

In some embodiments, the method further comprises selecting the subject for the clinical trial when the subject satisfies the inclusion criterion. In some embodiments, the method further comprises obtaining informed consent from the subject. the informed consent is obtained virtually using electronic signatures. In some embodiments, the informed consent is obtained for research use of the biological sample of the subject and data resulting therefrom. In some embodiments, the informed consent is obtained for access to at least a portion of medical records of the subject. In some embodiments, the informed consent comprises authorization for a health care provider to release the medical records of the subject. In some embodiments, the method further comprises excluding the subject for the clinical trial when the subject satisfies the exclusion criterion.

In some embodiments, the collection of data comprises assaying a biological sample of the subject. In some embodiments, the biological sample is selected from the group consisting of a blood sample, a urine sample, a stool sample, a saliva sample, a vaginal sample, a cervical sample, and a swab sample. In some embodiments, the biological sample comprises the blood sample. In some embodiments, the collection of data comprises shipping a biological sample collection kit to the location, and receiving a return shipment of the collected biological sample from the location. In some embodiments, the blood sample is obtained from the subject by a phlebotomy service. In some embodiments, the phlebotomy service is a mobile phlebotomy service. In some embodiments, the collection of data comprises shipping a biological sample collection kit to a phlebotomist, and receiving a return shipment of the collected biological sample from the phlebotomist. In some embodiments, the location is a residence, a workplace, or another location of choice of the subject.

In some embodiments, the clinical trial is an interventional clinical trial. In some embodiments, the interventional clinical trial is directed at least in part through a telemedicine consultation with a health care provider of the subject. In some embodiments, the clinical trial is an observational clinical trial. In some embodiments, the clinical trial is a longitudinal clinical trial. In some embodiments, the clinical trial is an Institutional Review Board (IRB) approved clinical trial.

Another aspect of the present disclosure provides a system comprising one or more computer processors that are individually or collectively programmed to implement a method for directing a clinical trial, the method comprising: (a) evaluating a subject for participation in the clinical trial, wherein the clinical trial is for assessing pregnancy-related states of subjects or fetuses thereof; (b) selecting the subject for the clinical trial, based at least in part on the evaluating in (a); and (c) directing collection of a biological sample of the subject at a location remote with respect to a health care facility.

Another aspect of the present disclosure provides a system comprising one or more computer processors that are individually or collectively programmed to implement a method for directing a clinical trial, the method comprising: (a) evaluating a subject for participation in the clinical trial, wherein the clinical trial is for assessing pregnancy-related states of fetuses of subjects; and (b) selecting the subject for the clinical trial, based at least in part on the evaluating in (a), wherein the clinical trial comprises collection of data in absence of the subject visiting a health care facility.

Another aspect of the present disclosure provides a system comprising one or more computer processors that are individually or collectively programmed to implement a method for directing a clinical trial, the method comprising: (a) evaluating a subject for participation in the clinical trial, wherein the clinical trial is for assessing pregnancy-related states of subjects or fetuses thereof, wherein the subjects are in a first trimester or a second trimester of pregnancy; and (b) selecting the subject for the clinical trial, based at least in part on the evaluating in (a), wherein the clinical trial comprises collection of data in absence of the subject visiting a health care facility.

Another aspect of the present disclosure provides a non-transitory computer-readable medium comprising machine-executable code that, upon execution by one or more computer processors, implements a method for directing a clinical trial, the method comprising: (a) evaluating a subject for participation in the clinical trial, wherein the clinical trial is for assessing pregnancy-related states of subjects or fetuses thereof; (b) selecting the subject for the clinical trial, based at least in part on the evaluating in (a); and (c) directing collection of a biological sample of the subject at a location remote with respect to a health care facility.

Another aspect of the present disclosure provides a non-transitory computer-readable medium comprising machine-executable code that, upon execution by one or more computer processors, implements a method for directing a clinical trial, the method comprising: (a) evaluating a subject for participation in the clinical trial, wherein the clinical trial is for assessing pregnancy-related states of fetuses of subjects; and (b) selecting the subject for the clinical trial, based at least in part on the evaluating in (a), wherein the clinical trial comprises collection of data in absence of the subject visiting a health care facility.

Another aspect of the present disclosure provides a non-transitory computer-readable medium comprising machine-executable code that, upon execution by one or more computer processors, implements a method for directing a clinical trial, the method comprising: (a) evaluating a subject for participation in the clinical trial, wherein the clinical trial is for assessing pregnancy-related states of subjects or fetuses thereof, wherein the subjects are in a first trimester or a second trimester of pregnancy; and (b) selecting the subject for the clinical trial, based at least in part on the evaluating in (a), wherein the clinical trial comprises collection of data in absence of the subject visiting a health care facility.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates an example workflow of a method for identifying or monitoring a pregnancy-related state of a subject, in accordance with disclosed embodiments.

FIG. 2 illustrates an example workflow of a method for identifying or monitoring a pregnancy-related state of a subject, in accordance with disclosed embodiments.

FIG. 3 illustrates an example workflow of a method for identifying or monitoring a pregnancy-related state of a subject, in accordance with disclosed embodiments.

FIG. 4 illustrates a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a”, “an”, and “the” include plural references unless the context clearly dictates otherwise. For example, the term “a nucleic acid” includes a plurality of nucleic acids, including mixtures thereof.

As used herein, the term “subject,” generally refers to an entity or a medium that has testable or detectable genetic information. A subject can be a person, individual, or patient. A subject can be a vertebrate, such as, for example, a mammal. Non-limiting examples of mammals include humans, simians, farm animals, sport animals, rodents, and pets. A subject can be a pregnant female subject. The subject can be a woman having a fetus (or multiple fetuses) or suspected of having the fetus (or multiple fetuses). The subject can be a person that is pregnant or is suspected of being pregnant. The subject may be displaying a symptom(s) indicative of a health or physiological state or condition of the subject, such as a pregnancy-related health or physiological state or condition of the subject. As an alternative, the subject can be asymptomatic with respect to such health or physiological state or condition.

The term “pregnancy-related state,” as used herein, generally refers to any health, physiological, and/or biochemical state or condition of a subject that is pregnant or is suspected of being pregnant, or of a fetus (or multiple fetuses) of the subject. Examples of pregnancy-related states include, without limitation, pre-term birth, full-term birth, gestational age, due date, onset of labor, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications, hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), fertility-related conditions, and fetal development stages or states (e.g., normal fetal organ function or development, and abnormal fetal organ function or development). For example, post-partum complications may include cardiovascular diseases (e.g., peripartum cardiomyopathy (PPCM), also known as postpartum cardiomyopathy), depression and anxiety (e.g., post-partum depression), post GDM (e.g., post-partum glucose tolerance to diabetes progression for a mother or infant), post-partum complications of preeclampsia (e.g., post-partum preeclampsia), excessive bleeding after giving birth (e.g., hemorrhage, placenta accreta), pulmonary embolism, cardiomyopathy, diabetes, anemia, and hypertensive disorders. In some situations, the pregnancy-related state is not associated with the health or physiological state or condition of a fetus (or multiple fetuses) of the subject.

As used herein, the term “sample,” generally refers to a biological sample obtained from or derived from one or more subjects. Biological samples may be cell-free biological samples or substantially cell-free biological samples, or may be processed or fractionated to produce cell-free biological samples. For example, cell-free biological samples may include cell-free ribonucleic acid (cfRNA), cell-free deoxyribonucleic acid (cfDNA), cell-free fetal DNA (cffDNA), plasma, serum, urine, saliva, amniotic fluid, and derivatives thereof. Cell-free biological samples may be obtained or derived from subjects using an ethylenediaminetetraacetic acid (EDTA) collection tube, a cell-free RNA collection tube (e.g., Streck), or a cell-free DNA collection tube (e.g., Streck). Biological samples may be derived from whole blood samples by fractionation. Biological samples or derivatives thereof may contain cells. For example, a biological sample may be a blood sample or a derivative thereof (e.g., blood collected by a collection tube or blood drops), a vaginal sample (e.g., a vaginal swab), or a cervical sample (e.g., a cervical swab).

As used herein, the term “nucleic acid” generally refers to a polymeric form of nucleotides of any length, either deoxyribonucleotides (dNTPs) or ribonucleotides (rNTPs), or analogs thereof. Nucleic acids may have any three-dimensional structure, and may perform any function, known or unknown. Non-limiting examples of nucleic acids include deoxyribonucleic (DNA), ribonucleic acid (RNA), coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent.

As used herein, the term “target nucleic acid” generally refers to a nucleic acid molecule in a starting population of nucleic acid molecules having a nucleotide sequence whose presence, amount, and/or sequence, or changes in one or more of these, are desired to be determined. A target nucleic acid may be any type of nucleic acid, including DNA, RNA, and analogs thereof. As used herein, a “target ribonucleic acid (RNA)” generally refers to a target nucleic acid that is RNA. As used herein, a “target deoxyribonucleic acid (DNA)” generally refers to a target nucleic acid that is DNA.

As used herein, the terms “amplifying” and “amplification” generally refer to increasing the size or quantity of a nucleic acid molecule. The nucleic acid molecule may be single-stranded or double-stranded. Amplification may include generating one or more copies or “amplified product” of the nucleic acid molecule. Amplification may be performed, for example, by extension (e.g., primer extension) or ligation. Amplification may include performing a primer extension reaction to generate a strand complementary to a single-stranded nucleic acid molecule, and in some cases generate one or more copies of the strand and/or the single-stranded nucleic acid molecule. The term “DNA amplification” generally refers to generating one or more copies of a DNA molecule or “amplified DNA product.” The term “reverse transcription amplification” generally refers to the generation of deoxyribonucleic acid (DNA) from a ribonucleic acid (RNA) template via the action of a reverse transcriptase.

### De-Centralized Pregnancy-Related Clinical Trials

Pregnancy-related complications such as pre-term birth are a leading cause of neonatal death and of complications later in life. Further, such pregnancy-related complications can cause negative health effects on maternal health. Clinical trials may be directed to identifying and monitoring pregnancy-related states of subjects or their fetuses using non-invasive and cost-effective approaches, toward improving maternal and fetal health.

Current pregnancy-related clinical trials may be inaccessible and incomplete. For cases in which pregnancies progress without pregnancy-related complications, limited methods of pregnancy monitoring may be available for a pregnancy subject, such as molecular tests, ultrasound imaging, and estimation of gestational age and/or due date using the last menstrual period. However, such monitoring methods may be complex, expensive, and unreliable. For example, molecular tests cannot predict gestational age, ultrasound imaging is expensive and best performed during the first trimester of pregnancy, and estimation of gestational age and/or due date using the last menstrual period can be unreliable. Further, for cases in which pregnancies progress with pregnancy-related complications such as risk of spontaneous pre-term delivery, the clinical utility of molecular tests, ultrasound imaging, and demographic factors may be limited. For example, molecular tests may have a limited BMI (body mass index) range, a limited gestational age and/or due date range (about 2 weeks), and a low positive predictive value (PPV); ultrasound imaging may be expensive and have low PPV and specificity; and the use of demographic factors to predict risk of pregnancy-related complications may be unreliable. Therefore, there exists an urgent clinical need for accurate pregnancy-related clinical trials for detection and monitoring of pregnancy-related states (e.g., estimation of gestational age, due date, and/or onset of labor, and prediction of pregnancy-related complications such as pre-term birth) toward clinically actionable outcomes.

The present disclosure provides methods and systems for directing pregnancy-related clinical trials. For example, the subjects may include subjects with one or more pregnancy-related states and subjects without pregnancy-related states. A method for directing a clinical trial may comprise evaluating a subject for participation in a clinical trial for assessing pregnancy-related states of subjects or fetuses thereof, selecting the subject for the clinical trial, based at least in part on the evaluating, and directing collection of a biological sample of the subject at a location remote with respect to a health care facility. A method for directing a clinical trial may comprise evaluating a subject for participation in a clinical trial for assessing pregnancy-related states of fetuses of subjects, and selecting the subject for the clinical trial, based at least in part on the evaluating, wherein the clinical trial comprises collection of data in absence of the subject visiting a health care facility. A method for directing a clinical trial may comprise evaluating a subject for participation in a clinical trial for assessing pregnancy-related states of subjects or fetuses thereof, wherein the subjects are in a first trimester or a second trimester of pregnancy, and selecting the subject for the clinical trial, based at least in part on the evaluating, wherein the clinical trial comprises collection of data in absence of the subject visiting a health care facility.

Pregnancy-related states may include, for example, pre-term birth, full-term birth, gestational age, due date, onset of labor, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications, hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, and macrosomia (large fetus for gestational age). In some embodiments, pregnancy-related states are not associated with the health of a fetus. In some embodiments, pregnancy-related states include neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), fertility-related conditions, and fetal development stages or states (e.g., normal fetal organ function or development, and abnormal fetal organ function or development). For example, post-partum complications may include cardiovascular diseases (e.g., peripartum cardiomyopathy (PPCM), also known as postpartum cardiomyopathy), depression and anxiety (e.g., post-partum depression), post GDM (e.g., post-partum glucose tolerance to diabetes progression for a mother or infant), post-partum complications of preeclampsia (e.g., post-partum preeclampsia), excessive bleeding after giving birth (e.g., hemorrhage, placenta accreta), pulmonary embolism, cardiomyopathy, diabetes, anemia, and hypertensive disorders.

In an aspect, the present disclosure provides a method for directing a clinical trial, comprising: (a) evaluating a subject for participation in the clinical trial, wherein the clinical trial is for assessing pregnancy-related states of subjects or fetuses thereof; (b) selecting the subject for the clinical trial, based at least in part on the evaluating in (a); and (c) directing collection of a biological sample of the subject at a location remote with respect to a health care facility.

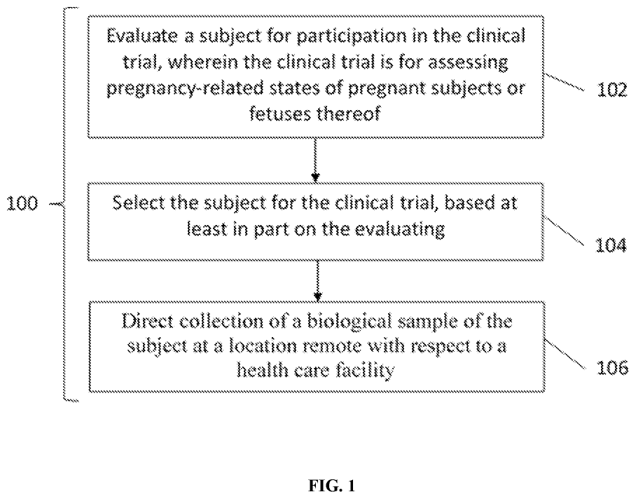

FIG. 1 illustrates an example workflow of a method for directing a clinical trial. In an aspect, the present disclosure provides a method 100 for directing a clinical trial. The method 100 may comprise evaluating a subject for participation in the clinical trial. In some embodiments, the clinical trial is for assessing pregnancy-related states of subjects or fetuses thereof (as in operation 102). Next, based at least in part on the evaluating, the method 100 may comprise selecting the subject for the clinical trial (as in operation 104). Next, the method 100 may comprise directing collection of a biological sample of the subject at a location remote with respect to a health care facility (as in operation 106).

In another aspect, the present disclosure provides a method for directing a clinical trial, comprising: (a) evaluating a subject for participation in the clinical trial, wherein the clinical trial is for assessing pregnancy-related states of fetuses of subjects; and (b) selecting the subject for the clinical trial, based at least in part on the evaluating in (a), wherein the clinical trial comprises collection of data in absence of the subject visiting a health care facility.

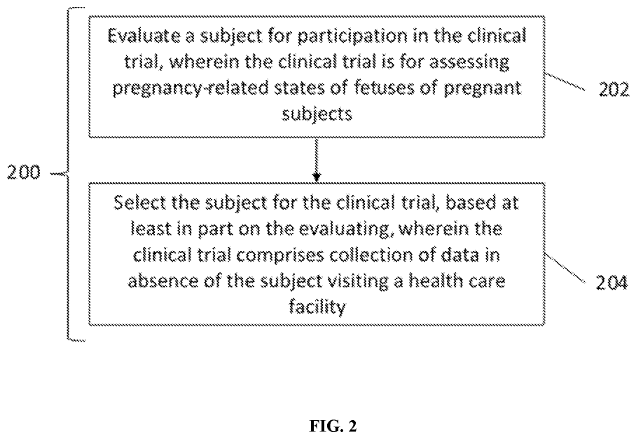

FIG. 2 illustrates an example workflow of a method for directing a clinical trial. In an aspect, the present disclosure provides a method 200 for directing a clinical trial. The method 200 may comprise evaluating a subject for participation in the clinical trial. In some embodiments, the clinical trial is for assessing pregnancy-related states of fetuses of subjects (as in operation 202). Next, based at least in part on the evaluating, the method 200 may comprise selecting the subject for the clinical trial (as in operation 204). In some embodiments, the clinical trial comprises collection of data in absence of the subject visiting a health care facility.

In another aspect, the present disclosure provides a method for directing a clinical trial, comprising: (a) evaluating a subject for participation in the clinical trial, wherein the clinical trial is for assessing pregnancy-related states of subjects or fetuses thereof, wherein the subjects are in a first trimester or a second trimester of pregnancy; and (b) selecting the subject for the clinical trial, based at least in part on the evaluating in (a), wherein the clinical trial comprises collection of data in absence of the subject visiting a health care facility.

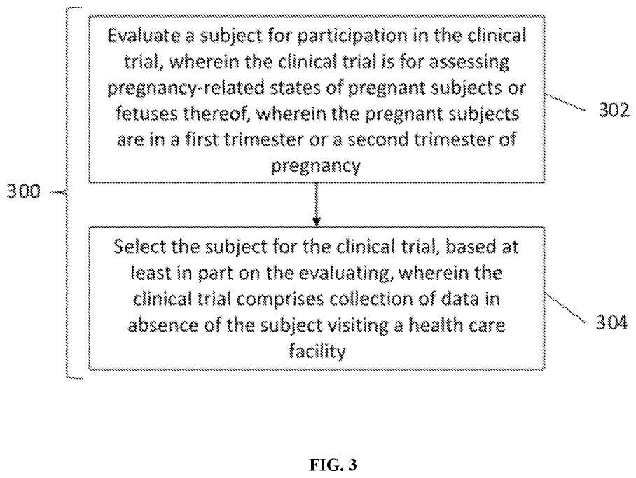

FIG. 3 illustrates an example workflow of a method for directing a clinical trial. In an aspect, the present disclosure provides a method 300 for directing a clinical trial. The method 300 may comprise evaluating a subject for participation in the clinical trial. In some embodiments, the clinical trial is for assessing pregnancy-related states of subjects or fetuses thereof (as in operation 302). In some embodiments, the subjects are in a first trimester or a second trimester of pregnancy. Next, based at least in part on the evaluating, the method 300 may comprise selecting the subject for the clinical trial (as in operation 304). In some embodiments, the clinical trial comprises collection of data in absence of the subject visiting a health care facility.

In some embodiments, the subject is pregnant or is suspected of being pregnant. For example, the subject may be pregnant (e.g., in a first trimester, second trimester, or third trimester of pregnancy). For example, the subject may be suspected of being pregnant (e.g., does not have a clinical indication of being pregnant, or is seeking a confirmation of a clinical indication of being pregnant). A clinical indication of being pregnant may comprise a blood test, a urine test (e.g., an immunoassay test), a hormone test (e.g., human chorionic gonadotropin (HCG)), an ultrasound scan (e.g., abdominal or transvaginal), pregnancy symptoms, etc.

In some embodiments, the subject is post-partum. In some examples, the subject is at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 4 months, at least about 5 months, at least about 6 months, at least about 7 months, at least about 8 months, at least about 9 months, at least about 10 months, at least about 11 months, or at least about 12 months post-partum. In some examples, the subject is at least about 1 year, at least about 1.5 years, at least about 2 years, at least about 2.5 years, at least about 3 years, at least about 3.5 years, at least about 4 years, at least about 4.5 years, or at least about 5 years post-partum.

The pregnancy-related state may comprise a pregnancy-related complication, such as pre-term birth, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications, hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), fertility-related conditions, and abnormal fetal development stages or states (e.g., abnormal fetal organ function or development). For example, post-partum complications may include cardiovascular diseases (e.g., peripartum cardiomyopathy (PPCM), also known as postpartum cardiomyopathy), depression and anxiety (e.g., post-partum depression), post GDM (e.g., post-partum glucose tolerance to diabetes progression for a mother or infant), post-partum complications of preeclampsia (e.g., post-partum preeclampsia), excessive bleeding after giving birth (e.g., hemorrhage, placenta accreta), pulmonary embolism, cardiomyopathy, diabetes, anemia, and hypertensive disorders.

The pregnancy-related state may comprise a full-term birth, normal fetal development stages or states (e.g., normal fetal organ function or development), or absence of a pregnancy-related complication (e.g., pre-term birth, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications, hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), fertility-related conditions, and abnormal fetal development stages or states (e.g., abnormal fetal organ function or development)). For example, post-partum complications may include cardiovascular diseases (e.g., peripartum cardiomyopathy (PPCM), also known as postpartum cardiomyopathy), depression and anxiety (e.g., post-partum depression), post GDM (e.g., post-partum glucose tolerance to diabetes progression for a mother or infant), post-partum complications of preeclampsia (e.g., post-partum preeclampsia), excessive bleeding after giving birth (e.g., hemorrhage, placenta accreta), pulmonary embolism, cardiomyopathy, diabetes, anemia, and hypertensive disorders.

The pregnancy-related state may comprise a quantitative assessment of pregnancy such as gestational age (e.g., measured in days, weeks or months) or due date (e.g., expressed as a predicted or estimated calendar date or range of calendar dates).

The pregnancy-related state may comprise a quantitative assessment of a pregnancy-related complication such as a likelihood, a susceptibility, or a risk (e.g., expressed as a probability, a relative probability, an odds ratio, or a risk score or risk index) of the pregnancy-related complication (e.g., pre-term birth, onset of labor, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications, hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), fertility-related conditions, and abnormal fetal development stages or states (e.g., abnormal fetal organ function or development)). For example, post-partum complications may include cardiovascular diseases (e.g., peripartum cardiomyopathy (PPCM), also known as postpartum cardiomyopathy), depression and anxiety (e.g., post-partum depression), post GDM (e.g., post-partum glucose tolerance to diabetes progression for a mother or infant), post-partum complications of preeclampsia (e.g., post-partum preeclampsia), excessive bleeding after giving birth (e.g., hemorrhage, placenta accreta), pulmonary embolism, cardiomyopathy, diabetes, anemia, and hypertensive disorders.

For example, the pregnancy-related state may comprise a likelihood or susceptibility of an onset of labor in the future (e.g., within about 1 hour, within about 2 hours, within about 4 hours, within about 6 hours, within about 8 hours, within about 10 hours, within about 12 hours, within about 14 hours, within about 16 hours, within about 18 hours, within about 20 hours, within about 22 hours, within about 24 hours, within about 1.5 days, within about 2 days, within about 2.5 days, within about 3 days, within about 3.5 days, within about 4 days, within about 4.5 days, within about days, within about 5.5 days, within about 6 days, within about 6.5 days, within about 7 days, within about 8 days, within about 9 days, within about 10 days, within about 12 days, within about 14 days, within about 3 weeks, within about 4 weeks, within about 5 weeks, within about 6 weeks, within about 7 weeks, within about 8 weeks, within about 9 weeks, within about 10 weeks, within about 11 weeks, within about 12 weeks, within about 13 weeks, or more than about 13 weeks).

In some embodiments, the pregnancy-related states of the subjects or the fetuses thereof comprise prenatal pregnancy-related states of the subjects or the fetuses thereof. Such prenatal pregnancy-related states of the subjects or the fetuses thereof may be present before a fetus of the subject is born. In some embodiments, the pregnancy-related states of the subjects or the fetuses thereof comprise postnatal pregnancy-related states of the subjects or the fetuses thereof. Such postnatal pregnancy-related states of the subjects or the fetuses thereof may be present after a fetus of the subject is born.

In some embodiments, the pregnancy-related states are selected from the group consisting of pre-term birth, full-term birth, gestational age, due date, onset of labor, a pregnancy-related hypertensive disorder, preeclampsia, eclampsia, gestational diabetes, a congenital disorder of a fetus of the subject, ectopic pregnancy, spontaneous abortion, stillbirth, a post-partum complication, hyperemesis gravidarum, hemorrhage or excessive bleeding during delivery, premature rupture of membrane, premature rupture of membrane in pre-term birth, placenta previa, intrauterine fetal growth restriction, macrosomia, a neonatal condition, a fertility-related condition, and a fetal development stage or state. In some embodiments, the pregnancy-related states are the pre-term birth. In some embodiments, the pregnancy-related states are the due date.

In some embodiments, the pregnancy-related state comprises at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, or more than 20 distinct pregnancy-related states. In some embodiments, the pregnancy-related state is assessed at each of a plurality of distinct time points (e.g., at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, or more than distinct time points).

Methods and systems of the present disclosure may perform pregnancy-related clinical trials in a de-centralized manner, such as using a direct-to-consumer or direct-to-patient approach. The clinical trials may be observational or interventional. For example, an observational clinical study may comprise evaluation, diagnosis, or prediction of health outcomes in a cohort of subjects (e.g., pregnant subjects) without manipulation or intervention by the clinical trial coordinators. For example, an interventional clinical trial may comprise evaluation of an intervention (e.g., a drug, a medical device, a clinical activity, or a clinical procedure) that is administered to a cohort of subjects (e.g., pregnant subjects). An interventional clinical trial may be directed at least in part through a telemedicine consultation with a health care provider of the subject. As another example, the clinical trial may be a longitudinal clinical trial. A clinical trial may be an international ethics review committee-approved clinical trial, for example, Institutional Review Board (IRB) approved clinical trial. A clinical trial may be a clinical trial approved by an international ethics committee that oversees human clinical trials, for example, a Research Ethics Committee (REC) approved clinical trial, a Medical Research Ethics Committee (MREC) approved clinical trial, or a Comite de Protection des Personnes (CPP) approved clinical trial, a Human Research Ethics Committee (HREC) approved clinical trial, a Etikprövningsnämnden approved clinical trial, or an Interagency Advisory Panel on Research Ethics approved clinical trial.

Methods and systems of the present disclosure may address issues with high expense and slow recruitment associated with clinical trials. Methods and systems of the present disclosure may comprise one or more of: a de-centralized direct-to-consumer (DTC) or direct-to-patient (DTP) model (which is performed at a plurality of distinct sites), clinical trial recruitment that includes digital marketing (e.g., through online recruitment), a fully virtual clinical trial in which subjects may complete their clinical trial activities in absence of visiting a clinical environment (e.g., while remaining in their home or workplace environment) or personal interaction with a clinical health provider (e.g., physician), and automatic integration of electronic health records (EHR) data of subjects. Methods and systems of the present disclosure may be used to direct pregnancy-related clinical trials using a direct-to-patient recruitment approach, which may enable creation of a new clinical site (MSA) to productivity within days, as compared to months using contract research organizations (CROs). Such pregnancy-related clinical trials may be tuned or targeted to achieve enrollment of a desired demographic or enrollment criteria mix of a site (MSA) and/or allow adjustment of enrollment rates by tuning or targeting incentives offered to subjects.

The DTC or DTP clinical trial may comprise digital recruitment of candidate subjects for the clinical trial through online channels (e.g., including providing informed consent and release of EHR data), collection of a biological sample or data of the subject at a location remote with respect to a health care facility (e.g., including scheduling of home visits by a phlebotomist or other sample collection vendor), transmission of the biological sample or data to a laboratory site, and analysis of the biological sample or data by clinical trial researchers. Subjects may be compensated for their time and effort in participation in the clinical trial (e.g., filling out forms, providing consent to provide EHR data, providing biological samples, and providing updates throughout the course of a pregnancy including post-natal periods).

The EHR data may include health data from subjects who are pregnant or suspected of being pregnant, who are in a first trimester, second trimester, or third trimester of pregnancy, who are not yet pregnant, or who have recently given birth. The EHR data (e.g., test results, imaging, and clinical notes) may be automatically synthesized or collated from a plurality of different sources, parsed, annotated, labeled, analyzed, and/or integrated into a database for evaluation of pregnancy-related states of subjects or fetuses thereof. For example, EHR data may be synthesized or collated from a plurality of different sources having different customized data formats, which may be parsed, annotated, labeled, analyzed, and/or integrated into a database for evaluation of pregnancy-related states of subjects or fetuses thereof. The EHR data may be retrieved, processed, and stored while adhering to regulatory compliance, such as compliance with HIPAA (Health Insurance Portability and Accountability Act of 1996), and data security standards (e.g., encryption). The EHR data may be parsed, annotated, labeled, analyzed, and/or integrated into a database in a format that is suitable for downstream bioinformatics analyses. For example, a plurality of pre-defined features may be automatically extracted (e.g., using machine learning algorithms such as natural language processing) from EHR data collected from each of a plurality of pregnant subjects in a cohort. In some embodiments, EHR data is obtained through a third-party medical record retrieval collection service. In some embodiments, the third-party is a data collection company or a data collection agency in the health malpractice field.

In some embodiments, the method further comprises recruiting the subject for participation in the clinical trial. In some embodiments, the recruiting comprises use of a digital marketing campaign (e.g., through Internet advertisements, e-mail, social media networks, SMS or MMS texts, etc.). In some embodiments, the recruiting comprises displaying advertisements to the subject through a computer network. In some embodiments, the advertisements are displayed or viewed through a social media channel, social networking (e.g., Facebook, Twitter, LinkedIn, Instagram, etc.), pregnancy education, pregnancy tracker, menstruation tracker, ovulation tracker, or fertility tracker platform. For example, the advertisements may be displayed through a mobile device (e.g., a smartphone, a laptop, a tablet computer, a smartwatch, or a combination thereof) of the subject. For example, advertisements may be displayed to a targeted group of candidate subjects based on a variety of demographic and interest group categories (e.g., age, race, education level, income, socioeconomic status, geographic location, family size, individuals who are pregnant, individuals who are trying to become pregnant, individuals who are seeking or receiving fertility treatments). For example, the advertisements may be geotargeted to specific geographic locations (e.g., zip codes). As another example, the advertisements may be targeted to subjects in additional metropolitan service areas with the ability for same day enrollment. As another example, the advertisements may be designed to build digital audiences that prioritize finding pregnant women (e.g., with specific sub-audiences for fertility, new mothers, and/or repeat mother targets). In some embodiments, the advertisements are adjusted in real time for varying incentive payment levels and subject characteristics (e.g., gestational age in weeks).

In some embodiments, recruiting the subject comprises, responsive to the subject responding to the displayed advertisement, directing the subject to visit or view one or more web pages of a user experience (UX) journey. For example, the web pages may provide information about the clinical trial, eligibility criteria of the clinical trial, and fillable forms for obtaining information from the subject. For example, the subject may respond to the displayed advertisement by clicking a hyperlink of the displayed advertisement. In some embodiments, recruiting the subject comprises displaying a contact form to the subject for completing, and receiving the completed contact form comprising personal contact information from the subject.

In some embodiments, the method further comprises displaying an eligibility questionnaire or a set of eligibility criteria to the subject via the web pages. In some embodiments, the method further comprises collecting response data from the subject using the eligibility questionnaire, a self-reported family history survey, a demographic survey, a medical condition survey, a customer satisfaction survey, or a combination thereof. In some embodiments, the method further comprises collecting EHR data of the subject, or receiving consent to receive EHR data of the subject from another source (e.g., a clinical database). For example, EHR data of the subject may be collected directly from the subject (e.g., through web browser forms), from laboratory testing of biological samples from the subject, retrieved from clinical databases, a third-party database, or a combination thereof.

In some embodiments, evaluating the subject for participation in the clinical trial comprises processing the response data collected from the subject. In some embodiments, processing the response data collected from the subject comprises determining a gestational age of the fetus of the subject and/or a time window for the collection of the biological sample of the subject. For example, the response data collected from the subject may comprise responses to questionnaires regarding eligibility for the clinical trial. In some embodiments, evaluating the subject for participation in the clinical trial comprises applying an inclusion criterion or an exclusion criterion of the clinical trial. In some embodiments, the inclusion criterion or the exclusion criterion is related to age, race, ethnicity, body mass index, gestational age, or location of the subject or a fetus of the subject. For example, certain categories or ranges may be selected for inclusion or exclusion for a given clinical trial.

In some embodiments, evaluating the subject for participation in the clinical trial comprises applying inclusion criterion or exclusion criterion of each of a plurality of clinical trials, so that the subject is evaluated for each of the plurality of clinical trials. For example, a given candidate subject may be evaluated for at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more than 100 different clinical trials based on response data related to eligibility criteria.

In some embodiments, the method further comprises selecting the subject for the clinical trial when the subject satisfies the inclusion criterion. For example, the subject may satisfy the inclusion criteria for one or more of a plurality of clinical trials. In some embodiments, the inclusion criteria are related to age, race, ethnicity, body mass index, gestational age, or location of the subject or a fetus of the subject. For example, certain categories or ranges may be selected for inclusion for a given clinical trial. The one or more clinical trials to which a given candidate subject has matched may be presented in a list (e.g., which may be ranked according to a suitability or match score).

In some embodiments, the method further comprises obtaining informed consent from the subject to be enrolled into a particular clinical trial, upon selecting the subject for the clinical trial. For example, the informed consent may be obtained virtually using electronic signatures. For example, the informed consent may be obtained for research use of the biological sample of the subject and data resulting therefrom. For example, the informed consent may be obtained for access to at least a portion of medical records of the subject. For example, the informed consent may comprise authorization for a health care provider to release the medical records of the subject.

In some embodiments, the method further comprises excluding the subject for the clinical trial when the subject satisfies the exclusion criterion. For example, the subject may satisfy the exclusion criteria for one or more of a plurality of clinical trials. In some embodiments, the exclusion criteria are related to age, race, ethnicity, body mass index, gestational age, or location of the subject or a fetus of the subject. For example, certain categories or ranges may be selected for exclusion for a given clinical trial.

### Assaying Biological Samples

Methods and systems of the present disclosure may comprise collection of biological samples from subjects and analysis of the biological samples. In some embodiments, the biological sample may be obtained from a subject with a pregnancy-related state (e.g., a pregnancy-related complication), from a subject that is suspected of having a pregnancy-related state (e.g., a pregnancy-related complication), or from a subject that does not have or is not suspected of having the pregnancy-related state (e.g., a pregnancy-related complication).

In some embodiments, the biological sample is selected from the group consisting of a blood sample, a urine sample, a stool sample, a saliva sample, a vaginal sample, a cervical sample, and a swab sample. In some embodiments, the biological sample comprises the blood sample. In some embodiments, the blood sample is obtained from the subject by a phlebotomy service (e.g., a mobile phlebotomy service). In some embodiments, a biological sample collection kit is shipped to the location, and a return shipment of the collected biological sample is received from the location. In some embodiments, the location is a residence, a workplace, or another location of choice of the subject. In some embodiments, a biological sample collection kit is shipped to a phlebotomist, and a return shipment of the collected biological sample is received from the phlebotomist.

The biological samples may be obtained or derived from a human subject (e.g., a pregnant female subject). The biological samples may be stored in a variety of storage conditions before processing, such as different temperatures (e.g., at room temperature, under refrigeration or freezer conditions, at about 25° C., at about 4° C., at about −18° C., about −20° C., or at about −80° C.) or different suspensions (e.g., blood collection tubes, EDTA collection tubes, cell-free RNA collection tubes, cell-free DNA collection tubes, urine sample collection containers, stool sample collection containers, saliva sample collection tube).

The biological sample may be taken before and/or after treatment of a subject with the pregnancy-related complication. Biological samples may be obtained from a subject during a treatment or a treatment regime. Multiple biological samples may be obtained from a subject to monitor the effects of the treatment over time. The biological sample may be taken from a subject known or suspected of having a pregnancy-related state (e.g., pregnancy-related complication) for which a definitive positive or negative diagnosis is not available via clinical tests. The biological sample may be taken from a subject suspected of having a pregnancy-related complication. The biological sample may be taken from a subject experiencing unexplained symptoms, such as fatigue, nausea, weight loss, aches and pains, weakness, or bleeding. The biological sample may be taken from a subject having explained symptoms. The biological sample may be taken from a subject at risk of developing a pregnancy-related complication due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

The biological sample may contain one or more analytes capable of being assayed, such as nucleic acids, proteins, metabolites, or a mixture, derivative, or combination thereof. Such nucleic acids may be deoxyribonucleic acid (DNA) or ribonucleic acid (RNA) molecules, which may be cell-free DNA (cfDNA) or cell-free RNA (cfRNA) molecules. For example, RNA or cfRNA molecules may be suitable for assaying to generate transcriptomic data. As another example, DNA or cfDNA molecules may be suitable for assaying to generate genomic data. As another example, proteins may be suitable for assaying to generate proteomic data. As another example, metabolites may be suitable for assaying to generate metabolomic data. One or more such analytes (e.g., DNA, cfDNA, RNA, cfRNA, proteins, or metabolites) may be isolated, extracted, or derived from one or more biological samples of a subject for downstream assaying using one or more suitable assays. For example, complementary DNA (cDNA) may be derived from RNA by performing reverse transcription.

After obtaining a biological sample from the subject, the biological sample may be processed to generate datasets indicative of a pregnancy-related state of the subject. For example, a presence, absence, or quantitative assessment of nucleic acid molecules of the biological sample at a panel of pregnancy-related genomic loci (e.g., quantitative measures of RNA transcripts or DNA at the pregnancy-related genomic loci), proteomic data comprising quantitative measures of proteins of the dataset at a panel of pregnancy-related proteins, and/or metabolome data comprising quantitative measures of a panel of pregnancy-related metabolites may be indicative of a pregnancy-related state. Processing the biological sample obtained from the subject may comprise (i) subjecting the biological sample to conditions that are sufficient to isolate, enrich, or extract a plurality of nucleic acid molecules, proteins, and/or metabolites, and (ii) assaying the plurality of nucleic acid molecules, proteins, and/or metabolites to generate the dataset.

In some embodiments, a plurality of nucleic acid molecules is extracted from the biological sample and subjected to sequencing to generate a plurality of sequencing reads. The nucleic acid molecules may comprise ribonucleic acid (RNA) or deoxyribonucleic acid (DNA). The nucleic acid molecules (e.g., RNA or DNA) may be extracted from the biological sample by a variety of methods, such as a FastDNA Kit protocol from MP Biomedicals, a QIAamp DNA cell-free biological mini kit from Qiagen, or a cell-free biological DNA isolation kit protocol from Norgen Biotek. The extraction method may extract all RNA or DNA molecules from a sample. Alternatively, the extract method may selectively extract a portion of RNA or DNA molecules from a sample. Extracted RNA molecules from a sample may be converted to DNA molecules by reverse transcription (RT).

The sequencing may be performed by any suitable sequencing methods, such as massively parallel sequencing (MPS), paired-end sequencing, high-throughput sequencing, next-generation sequencing (NGS), shotgun sequencing, single-molecule sequencing, nanopore sequencing, semiconductor sequencing, pyrosequencing, sequencing-by-synthesis (SBS), sequencing-by-ligation, sequencing-by-hybridization, and RNA-Seq (Illumina).

The sequencing may comprise nucleic acid amplification (e.g., of RNA or DNA molecules). In some embodiments, the nucleic acid amplification is polymerase chain reaction (PCR). A suitable number of rounds of PCR (e.g., PCR, qPCR, reverse-transcriptase PCR, digital PCR, etc.) may be performed to sufficiently amplify an initial amount of nucleic acid (e.g., RNA or DNA) to a desired input quantity for subsequent sequencing. In some cases, the PCR may be used for global amplification of target nucleic acids. This may comprise using adapter sequences that may be first ligated to different molecules followed by PCR amplification using universal primers. PCR may be performed using any of a number of commercial kits, e.g., provided by Life Technologies, Affymetrix, Promega, Qiagen, etc. In other cases, only certain target nucleic acids within a population of nucleic acids may be amplified. Specific primers, possibly in conjunction with adapter ligation, may be used to selectively amplify certain targets for downstream sequencing. The PCR may comprise targeted amplification of one or more genomic loci, such as genomic loci associated with pregnancy-related states. The sequencing may comprise use of simultaneous reverse transcription (RT) and polymerase chain reaction (PCR), such as a OneStep RT-PCR kit protocol by Qiagen, NEB, Thermo Fisher Scientific, or Bio-Rad.

RNA or DNA molecules isolated or extracted from a biological sample may be tagged, e.g., with identifiable tags, to allow for multiplexing of a plurality of samples. Any number of RNA or DNA samples may be multiplexed. For example a multiplexed reaction may contain RNA or DNA from at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 25, at least about 30, at least about 35, at least about at least about 45, at least about 50, at least about 55, at least about 60, at least about 65, at least about 70, at least about 75, at least about 80, at least about 85, at least about 90, at least about 95, at least about 100, or more than 100 initial biological samples. For example, a plurality of biological samples may be tagged with sample barcodes such that each DNA molecule may be traced back to the sample (and the subject) from which the DNA molecule originated. Such tags may be attached to RNA or DNA molecules by ligation or by PCR amplification with primers.

After subjecting the nucleic acid molecules to sequencing, suitable bioinformatics processes may be performed on the sequence reads to generate the data indicative of the presence, absence, or relative assessment of the pregnancy-related state. For example, the sequence reads may be aligned to one or more reference genomes (e.g., a genome of one or more species such as a human genome). The aligned sequence reads may be quantified at one or more genomic loci to generate the datasets indicative of the pregnancy-related state. For example, quantification of sequences corresponding to a plurality of genomic loci associated with pregnancy-related states may generate the datasets indicative of the pregnancy-related state.

The biological sample may be processed without any nucleic acid extraction. For example, the pregnancy-related state may be identified or monitored in the subject by using probes configured to selectively enrich nucleic acid (e.g., RNA or DNA) molecules corresponding to the plurality of pregnancy-related genomic loci. The probes may be nucleic acid primers. The probes may have sequence complementarity with nucleic acid sequences from one or more of the plurality of pregnancy-related genomic loci or genomic regions. The plurality of pregnancy-related genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about at least about 70, at least about 75, at least about 80, at least about 85, at least about 90, at least about 95, at least about 100, or more distinct pregnancy-related genomic loci or genomic regions.

The pregnancy-related genomic loci or genomic regions may be associated with gestational age, pre-term birth, due date, onset of labor, or other pregnancy-related states or complications, such as the genomic loci described by, for example, Ngo et al. (“Noninvasive blood tests for fetal development predict gestational age and preterm delivery,” Science, 360(6393), pp. 1133-1136, 8 Jun. 2018), which is incorporated by reference herein in its entirety.

The probes may be nucleic acid molecules (e.g., RNA or DNA) having sequence complementarity with nucleic acid sequences (e.g., RNA or DNA) of the one or more genomic loci (e.g., pregnancy-related genomic loci). These nucleic acid molecules may be primers or enrichment sequences. The assaying of the biological sample using probes that are selective for the one or more genomic loci (e.g., pregnancy-related genomic loci) may comprise use of array hybridization (e.g., microarray-based), polymerase chain reaction (PCR), or nucleic acid sequencing (e.g., RNA sequencing or DNA sequencing). In some embodiments, DNA or RNA may be assayed by one or more of: isothermal DNA/RNA amplification methods (e.g., loop-mediated isothermal amplification (LAMP), helicase dependent amplification (HDA), rolling circle amplification (RCA), recombinase polymerase amplification (RPA)), immunoassays, electrochemical assays, surface-enhanced Raman spectroscopy (SERS), quantum dot (QD)-based assays, molecular inversion probes, droplet digital PCR (ddPCR), CRISPR/Cas-based detection (e.g., CRISPR-typing PCR (ctPCR), specific high-sensitivity enzymatic reporter un-locking (SHERLOCK), DNA endonuclease targeted CRISPR trans reporter (DETECTR), and CRISPR-mediated analog multi-event recording apparatus (CAMERA)), and laser transmission spectroscopy (LTS).

The assay readouts may be quantified at one or more genomic loci (e.g., pregnancy-related genomic loci) to generate the data indicative of the pregnancy-related state. For example, quantification of array hybridization or polymerase chain reaction (PCR) corresponding to a plurality of genomic loci (e.g., pregnancy-related genomic loci) may generate data indicative of the pregnancy-related state. Assay readouts may comprise quantitative PCR (qPCR) values, digital PCR (dPCR) values, digital droplet PCR (ddPCR) values, fluorescence values, etc., or normalized values thereof. The assay may be a home use test configured to be performed in a home setting.

In some embodiments, multiple assays are used to process biological samples of a subject. For example, a first assay may be used to process a first biological sample obtained or derived from the subject to generate a first dataset; and based at least in part on the first dataset, a second assay different from said first assay may be used to process a second biological sample obtained or derived from the subject to generate a second dataset indicative of said pregnancy-related state. The first assay may be used to screen or process biological samples of a set of subjects, while the second or subsequent assays may be used to screen or process biological samples of a smaller subset of the set of subjects. The first assay may have a low cost and/or a high sensitivity of detecting one or more pregnancy-related states (e.g., pregnancy-related complication), that is amenable to screening or processing biological samples of a relatively large set of subjects. The second assay may have a higher cost and/or a higher specificity of detecting one or more pregnancy-related states (e.g., pregnancy-related complication), that is amenable to screening or processing biological samples of a relatively small set of subjects (e.g., a subset of the subjects screened using the first assay). The second assay may generate a second dataset having a specificity (e.g., for one or more pregnancy-related states such as pregnancy-related complications) greater than the first dataset generated using the first assay. As an example, one or more biological samples may be processed using a cfRNA assay on a large set of subjects and subsequently a metabolomics assay on a smaller subset of subjects, or vice versa. The smaller subset of subjects may be selected based at least in part on the results of the first assay.

Alternatively, multiple assays may be used to simultaneously process biological samples of a subject. For example, a first assay may be used to process a first biological sample obtained or derived from the subject to generate a first dataset indicative of the pregnancy-related state; and a second assay different from the first assay may be used to process a second biological sample obtained or derived from the subject to generate a second dataset indicative of the pregnancy-related state. Any or all of the first dataset and the second dataset may then be analyzed to assess the pregnancy-related state of the subject. For example, a single diagnostic index or diagnosis score can be generated based on a combination of the first dataset and the second dataset. As another example, separate diagnostic indexes or diagnosis scores can be generated based on the first dataset and the second dataset.

The biological samples may be processed using a metabolomics assay. For example, a metabolomics assay can be used to identify a quantitative measure (e.g., indicative of a presence, absence, or relative amount) of each of a plurality of pregnancy-related metabolites in a biological sample of the subject. The metabolomics assay may be configured to process biological samples such as a blood sample or a urine sample (or derivatives thereof) of the subject. A quantitative measure (e.g., indicative of a presence, absence, or relative amount) of pregnancy-related metabolites in the biological sample may be indicative of one or more pregnancy-related states. The metabolites in the biological sample may be produced (e.g., as an end product or a byproduct) as a result of one or more metabolic pathways corresponding to pregnancy-related genes. Assaying one or more metabolites of the biological sample may comprise isolating or extracting the metabolites from the biological sample. The metabolomics assay may be used to generate datasets indicative of the quantitative measure (e.g., indicative of a presence, absence, or relative amount) of each of a plurality of pregnancy-related metabolites in the biological sample of the subject.

The metabolomics assay may analyze a variety of metabolites in the biological sample, such as small molecules, lipids, amino acids, peptides, nucleotides, hormones and other signaling molecules, cytokines, minerals and elements, polyphenols, fatty acids, dicarboxylic acids, alcohols and polyols, alkanes and alkenes, keto acids, glycolipids, carbohydrates, hydroxy acids, purines, prostanoids, catecholamines, acyl phosphates, phospholipids, cyclic amines, amino ketones, nucleosides, glycerolipids, aromatic acids, retinoids, amino alcohols, pterins, steroids, carnitines, leukotrienes, indoles, porphyrins, sugar phosphates, coenzyme A derivatives, glucuronides, ketones, sugar phosphates, inorganic ions and gases, sphingolipids, bile acids, alcohol phosphates, amino acid phosphates, aldehydes, quinones, pyrimidines, pyridoxals, tricarboxylic acids, acyl glycines, cobalamin derivatives, lipoamides, biotin, and polyamines.

The metabolomics assay may comprise, for example, one or more of: mass spectroscopy (MS), targeted MS, gas chromatography (GC), high performance liquid chromatography (HPLC), capillary electrophoresis (CE), nuclear magnetic resonance (NMR) spectroscopy, ion-mobility spectrometry, Raman spectroscopy, electrochemical assay, or immune assay.

The biological samples may be processed using a methylation-specific assay. For example, a methylation-specific assay can be used to identify a quantitative measure (e.g., indicative of a presence, absence, or relative amount) of methylation each of a plurality of pregnancy-related genomic loci in a biological sample of the subject. The methylation-specific assay may be configured to process biological samples such as a blood sample or a urine sample (or derivatives thereof) of the subject. A quantitative measure (e.g., indicative of a presence, absence, or relative amount) of methylation of pregnancy-related genomic loci in the biological sample may be indicative of one or more pregnancy-related states. The methylation-specific assay may be used to generate datasets indicative of the quantitative measure (e.g., indicative of a presence, absence, or relative amount) of methylation of each of a plurality of pregnancy-related genomic loci in the biological sample of the subject.

The methylation-specific assay may comprise, for example, one or more of: a methylation-aware sequencing (e.g., using bisulfite treatment), pyrosequencing, methylation-sensitive single-strand conformation analysis (MS-SSCA), high-resolution melting analysis (FIRM), methylation-sensitive single-nucleotide primer extension (MS-SnuPE), base-specific cleavage/MALDI-TOF, microarray-based methylation assay, methylation-specific PCR, targeted bisulfite sequencing, oxidative bisulfite sequencing, mass spectroscopy-based bisulfite sequencing, or reduced representation bisulfite sequence (RRBS).

The biological samples may be processed using a proteomics assay. For example, a proteomics assay can be used to identify a quantitative measure (e.g., indicative of a presence, absence, or relative amount) of each of a plurality of pregnancy-related proteins or polypeptides in a biological sample of the subject. The proteomics assay may be configured to process biological samples such as a blood sample or a urine sample (or derivatives thereof) of the subject. A quantitative measure (e.g., indicative of a presence, absence, or relative amount) of pregnancy-related proteins or polypeptides in the biological sample may be indicative of one or more pregnancy-related states. The proteins or polypeptides in the biological sample may be produced (e.g., as an end product or a byproduct) as a result of one or more biochemical pathways corresponding to pregnancy-related genes. Assaying one or more proteins or polypeptides of the biological sample may comprise isolating or extracting the proteins or polypeptides from the biological sample. The proteomics assay may be used to generate datasets indicative of the quantitative measure (e.g., indicative of a presence, absence, or relative amount) of each of a plurality of pregnancy-related proteins or polypeptides in the biological sample of the subject.

The proteomics assay may analyze a variety of proteins or polypeptides in the biological sample, such as proteins made under different cellular conditions (e.g., development, cellular differentiation, or cell cycle). The proteomics assay may comprise, for example, one or more of: an antibody-based immunoassay, an Edman degradation assay, a mass spectrometry-based assay (e.g., matrix-assisted laser desorption/ionization (MALDI) and electrospray ionization (ESI)), a top-down proteomics assay, a bottom-up proteomics assay, a mass spectrometric immunoassay (MSIA), a stable isotope standard capture with anti-peptide antibodies (SISCAPA) assay, a fluorescence two-dimensional differential gel electrophoresis (2-D DIGE) assay, a quantitative proteomics assay, a protein microarray assay, or a reverse-phased protein microarray assay. The proteomics assay may detect post-translational modifications of proteins or polypeptides (e.g., phosphorylation, ubiquitination, methylation, acetylation, glycosylation, oxidation, and nitrosylation). The proteomics assay may identify or quantify one or more proteins or polypeptides from a database (e.g., Human Protein Atlas, PeptideAtlas, and UniProt).

### Kits

The present disclosure provides kits that may be used to obtain or collect biological samples from a subject (e.g., a pregnant subject). A kit may comprise one or more components for biological sample collection, such as a blood collection tube (BCT), an ethylenediaminetetraacetic acid (EDTA) collection tube, a cell-free RNA collection tube (e.g., Streck), a cell-free DNA collection tube (e.g., Streck), a vaginal swab, a cervical swab, a cheek swab, a urine collection container, a stool collection container, or a finger prick device and blood drop collection card. The components for biological sample collection may comprise a preservative (e.g., to preserve a collected biological sample) or temperature control (e.g., cold gel pack, frozen gel pack, dry ice). The kit may comprise a shipping container to enclose the collected biological sample and a shipping label for return shipping. The kit may comprise instructions for using the one or more components for biological sample collection. The instructions may be used by the subject and/or a biological sample collection specialist (e.g., a phlebotomist).

The present disclosure provides kits that may be used to evaluate, identify, or monitor a pregnancy-related state of a subject (e.g., as part of a clinical trial of subjects). A kit may comprise probes for identifying a quantitative measure of nucleic acid sequences corresponding to pregnancy-related genomic loci in a biological sample of the subject. A quantitative measure (e.g., indicative of a presence, absence, or relative amount) of nucleic acid sequences corresponding to pregnancy-related genomic loci in the biological sample may be indicative of one or more pregnancy-related states. The probes may be selective for the nucleic acid sequences corresponding to pregnancy-related genomic loci in the biological sample. A kit may comprise instructions for using the probes to process the biological sample to generate datasets indicative of a quantitative measure (e.g., indicative of a presence, absence, or relative amount) of nucleic acid sequences corresponding to pregnancy-related genomic loci in the biological sample.

The probes in the kit may be selective for the nucleic acid sequences corresponding to pregnancy-related genomic loci in the biological sample. The probes in the kit may be configured to selectively enrich nucleic acid (e.g., RNA or DNA) molecules corresponding to the pregnancy-related genomic loci. The probes in the kit may be nucleic acid primers. The probes in the kit may have sequence complementarity with nucleic acid sequences corresponding to one or more of the pregnancy-related genomic loci or genomic regions. The plurality of pregnancy-related genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more distinct pregnancy-related genomic loci or genomic regions.

The instructions in the kit may comprise instructions to assay the biological sample using the probes that are selective for the nucleic acid sequences corresponding to pregnancy-related genomic loci in the biological sample. These probes may be nucleic acid molecules (e.g., RNA or DNA) having sequence complementarity with nucleic acid sequences (e.g., RNA or DNA) corresponding to one or more of the pregnancy-related genomic loci or genomic regions. These nucleic acid molecules may be primers or enrichment sequences.

The instructions to assay the biological sample may comprise introductions to perform array hybridization, polymerase chain reaction (PCR), or nucleic acid sequencing (e.g., DNA sequencing or RNA sequencing) to process the biological sample to generate datasets indicative of a quantitative measure (e.g., indicative of a presence, absence, or relative amount) of nucleic acid sequences corresponding to one or more of the pregnancy-related genomic loci or genomic regions. A quantitative measure (e.g., indicative of a presence, absence, or relative amount) of nucleic acid sequences corresponding to one or more of the pregnancy-related genomic loci or genomic regions may be indicative of one or more pregnancy-related states.

The instructions in the kit may comprise instructions to measure and interpret assay readouts, which may be quantified at one or more of the pregnancy-related genomic loci to generate the datasets indicative of a quantitative measure (e.g., indicative of a presence, absence, or relative amount) of nucleic acid sequences corresponding to one or more of the pregnancy-related genomic loci or genomic regions. For example, quantification of array hybridization or polymerase chain reaction (PCR) corresponding to the plurality of pregnancy-related genomic loci may generate the datasets indicative of a quantitative measure (e.g., indicative of a presence, absence, or relative amount) of nucleic acid sequences corresponding to one or more of the pregnancy-related genomic loci or genomic regions. Assay readouts may comprise quantitative PCR (qPCR) values, digital PCR (dPCR) values, digital droplet PCR (ddPCR) values, fluorescence values, etc., or normalized values thereof.

A kit may comprise a proteomics assay for identifying a quantitative measure (e.g., indicative of a presence, absence, or relative amount) of pregnancy-related proteins, peptides, protein constituents, or amino acids in a biological sample of the subject. A quantitative measure (e.g., indicative of a presence, absence, or relative amount) of pregnancy-related proteins, peptides, protein constituents, or amino acids in the biological sample may be indicative of one or more pregnancy-related states. The proteins, peptides, protein constituents, or amino acids in the biological sample may be expressed or produced as a result of one or more pregnancy-related genes. A kit may comprise instructions for isolating or extracting the proteins, peptides, protein constituents, or amino acids from the biological sample and/or for using the proteomics assay to generate datasets indicative of the quantitative measure (e.g., indicative of a presence, absence, or relative amount) of pregnancy-related proteins, peptides, protein constituents, or amino acids in the biological sample of the subject.

A kit may comprise a metabolomics assay for identifying a quantitative measure (e.g., indicative of a presence, absence, or relative amount) of pregnancy-related metabolites in a biological sample of the subject. A quantitative measure (e.g., indicative of a presence, absence, or relative amount) of pregnancy-related metabolites in the biological sample may be indicative of one or more pregnancy-related states. The metabolites in the biological sample may be produced (e.g., as an end product or a byproduct) as a result of one or more metabolic pathways corresponding to pregnancy-related genes. A kit may comprise instructions for isolating or extracting the metabolites from the biological sample and/or for using the metabolomics assay to generate datasets indicative of the quantitative measure (e.g., indicative of a presence, absence, or relative amount) of pregnancy-related metabolites in the biological sample of the subject.

### Trained Algorithms

After using one or more assays to process one or more biological samples derived from the subject to generate one or more datasets indicative of the pregnancy-related state or pregnancy-related complication, a trained algorithm may be used to process one or more of the datasets (e.g., at each of a plurality of pregnancy-related genomic loci) to determine the pregnancy-related state. For example, the trained algorithm may be used to determine quantitative measures of sequences at each of the plurality of pregnancy-related genomic loci in the biological samples.

The trained algorithm may be configured to identify the pregnancy-related state with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99% for at least about 25, at least about 50, at least about 100, at least about 150, at least about 200, at least about 250, at least about 300, at least about 350, at least about 400, at least about 450, at least about 500, or more than about 500 independent samples.

The trained algorithm may comprise a supervised machine learning algorithm. The trained algorithm may comprise a classification and regression tree (CART) algorithm. The supervised machine learning algorithm may comprise, for example, a linear regression, a logistic regression, a ridge regression, a lasso regression, an elastic net regression, an ANOVA model, a naïve Bayes classifier, a Random Forest, a support vector machine (SVM), a neural network, a deep learning algorithm, or a combination thereof. The trained algorithm may comprise a differential expression algorithm. The differential expression algorithm may comprise a use comparison of stochastic models, generalized Poisson (GPseq), mixed Poisson (TSPM), Poisson log-linear (PoissonSeq), negative binomial (edgeR, DESeq, baySeq, NBPSeq), linear model fit by MAANOVA, or a combination thereof. The trained algorithm may comprise an unsupervised machine learning algorithm.

The trained algorithm may be configured to accept a plurality of input variables and to produce one or more output values based on the plurality of input variables. The plurality of input variables may comprise one or more datasets indicative of a pregnancy-related state. For example, an input variable may comprise a number of sequences corresponding to or aligning to each of the plurality of pregnancy-related genomic loci. The plurality of input variables may also include clinical health data of a subject.

The trained algorithm may comprise a classifier, such that each of the one or more output values comprises one of a fixed number of possible values (e.g., a linear classifier, a logistic regression classifier, etc.) indicating a classification of the biological sample by the classifier. The trained algorithm may comprise a binary classifier, such that each of the one or more output values comprises one of two values (e.g., {0, 1}, {positive, negative}, or {high-risk, low-risk}) indicating a classification of the biological sample by the classifier. The trained algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the biological sample by the classifier.

The output values may comprise descriptive labels, numerical values, or a combination thereof. Some of the output values may comprise descriptive labels. Such descriptive labels may provide an identification or indication of the disease or disorder state of the subject, and may comprise, for example, positive, negative, high-risk, intermediate-risk, low-risk, or indeterminate. Such descriptive labels may provide an identification of a treatment for the subject's pregnancy-related state, and may comprise, for example, a therapeutic intervention, a duration of the therapeutic intervention, and/or a dosage of the therapeutic intervention suitable to treat a pregnancy-related condition. Such descriptive labels may provide an identification of secondary clinical tests that may be appropriate to perform on the subject, and may comprise, for example, an imaging test, a blood test, a computed tomography (CT) scan, a magnetic resonance imaging (MRI) scan, an ultrasound scan, a chest X-ray, a positron emission tomography (PET) scan, a PET-CT scan, a biological cytology, an amniocentesis, a non-invasive prenatal test (NIPT), or any combination thereof. For example, such descriptive labels may provide a prognosis of the pregnancy-related state of the subject. As another example, such descriptive labels may provide a relative assessment of the pregnancy-related state (e.g., an estimated gestational age in number of days, weeks, or months) of the subject. Some descriptive labels may be mapped to numerical values, for example, by mapping “positive” to 1 and “negative” to 0.

Some of the output values may comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the pregnancy-related state of the subject. Some numerical values may be mapped to descriptive labels, for example, by mapping 1 to “positive” and 0 to “negative.”

Some of the output values may be assigned based on one or more cutoff values. For example, a binary classification of samples may assign an output value of “positive” or 1 if the sample indicates that the subject has at least a 50% probability of having a pregnancy-related state (e.g., pregnancy-related complication). For example, a binary classification of samples may assign an output value of “negative” or 0 if the sample indicates that the subject has less than a 50% probability of having a pregnancy-related state (e.g., pregnancy-related complication). In this case, a single cutoff value of 50% is used to classify samples into one of the two possible binary output values. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, a classification of samples may assign an output value of “positive” or 1 if the sample indicates that the subject has a probability of having a pregnancy-related state (e.g., pregnancy-related complication) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of samples may assign an output value of “positive” or 1 if the sample indicates that the subject has a probability of having a pregnancy-related state (e.g., pregnancy-related complication) of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classification of samples may assign an output value of “negative” or 0 if the sample indicates that the subject has a probability of having a pregnancy-related state (e.g., pregnancy-related complication) of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of having a pregnancy-related state (e.g., pregnancy-related complication) of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classification of samples may assign an output value of “indeterminate” or 2 if the sample is not classified as “positive”, “negative”, 1, or 0. In this case, a set of two cutoff values is used to classify samples into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values, where n is any positive integer.

The trained algorithm may be trained with a plurality of independent training samples. Each of the independent training samples may comprise a biological sample from a subject, associated datasets obtained by assaying the biological sample, and one or more known output values corresponding to the biological sample (e.g., a clinical diagnosis, prognosis, absence, or treatment efficacy of a pregnancy-related state of the subject). Independent training samples may comprise biological samples and associated datasets and outputs obtained or derived from a plurality of different subjects. Independent training samples may comprise biological samples and associated datasets and outputs obtained at a plurality of different time points from the same subject (e.g., on a regular basis such as weekly, biweekly, or monthly). Independent training samples may be associated with presence of the pregnancy-related state (e.g., training samples comprising biological samples and associated datasets and outputs obtained or derived from a plurality of subjects known to have the pregnancy-related state). Independent training samples may be associated with absence of the pregnancy-related state (e.g., training samples comprising biological samples and associated datasets and outputs obtained or derived from a plurality of subjects who are known to not have a previous diagnosis of the pregnancy-related state or who have received a negative test result for the pregnancy-related state).

In some embodiments, training samples may comprise clinical health data. For example, clinical health data may comprise one or more quantitative measures of the subject, such as age, weight, height, body mass index (BMI), blood pressure, heart rate, glucose levels, number of previous pregnancies, and number of previous births. As another example, the clinical health data can comprise one or more categorical measures, such as race, ethnicity, history of medication or other clinical treatment, history of tobacco use, history of alcohol consumption, daily activity or fitness level, genetic test results, blood test results, imaging results, and fetal screening results.

The trained algorithm may be trained with at least about 5, at least about 10, at least about at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise biological samples associated with presence of the pregnancy-related state and/or biological samples associated with absence of the pregnancy-related state. The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with presence of the pregnancy-related state. In some embodiments, the biological sample is independent of samples used to train the trained algorithm.

The trained algorithm may be trained with a first number of independent training samples associated with presence of the pregnancy-related state and a second number of independent training samples associated with absence of the pregnancy-related state. The first number of independent training samples associated with presence of the pregnancy-related state may be no more than the second number of independent training samples associated with absence of the pregnancy-related state. The first number of independent training samples associated with presence of the pregnancy-related state may be equal to the second number of independent training samples associated with absence of the pregnancy-related state. The first number of independent training samples associated with presence of the pregnancy-related state may be greater than the second number of independent training samples associated with absence of the pregnancy-related state.

The trained algorithm may be configured to identify the pregnancy-related state at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the pregnancy-related state by the trained algorithm may be calculated as the percentage of independent test samples (e.g., subjects known to have the pregnancy-related state or subjects with negative clinical test results for the pregnancy-related state) that are correctly identified or classified as having or not having the pregnancy-related state. The accuracy of identifying the pregnancy-related state may be determined using a percentage of a plurality of independent test samples corresponding to (1) a first plurality of subjects or fetuses of the first plurality of subjects having a pregnancy-related state relative to (2) a second plurality of subjects or fetuses of a second plurality of subjects who do not have the pregnancy-related state, that is correctly determined to have, not have, be at risk of having, or not be at risk of having the pregnancy-related state.

The trained algorithm may be configured to identify the pregnancy-related state with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the pregnancy-related state using the trained algorithm may be calculated as the percentage of biological samples identified or classified as having the pregnancy-related state that correspond to subjects that truly have the pregnancy-related state.

The trained algorithm may be configured to identify the pregnancy-related state with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the pregnancy-related state using the trained algorithm may be calculated as the percentage of biological samples identified or classified as not having the pregnancy-related state that correspond to subjects that truly do not have the pregnancy-related state.

The trained algorithm may be configured to identify the pregnancy-related state with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the pregnancy-related state using the trained algorithm may be calculated as the percentage of independent test samples associated with presence of the pregnancy-related state (e.g., subjects known to have the pregnancy-related state) that are correctly identified or classified as having the pregnancy-related state.

The trained algorithm may be configured to identify the pregnancy-related state with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the pregnancy-related state using the trained algorithm may be calculated as the percentage of independent test samples associated with absence of the pregnancy-related state (e.g., subjects with negative clinical test results for the pregnancy-related state) that are correctly identified or classified as not having the pregnancy-related state.

The trained algorithm may be configured to identify the pregnancy-related state with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the trained algorithm in classifying biological samples as having or not having the pregnancy-related state.

The trained algorithm may be adjusted or tuned to improve one or more of the performance, accuracy, PPV, NPV, clinical sensitivity, clinical specificity, or AUC of identifying the pregnancy-related state. The trained algorithm may be adjusted or tuned by adjusting parameters of the trained algorithm (e.g., a set of cutoff values used to classify a biological sample as described elsewhere herein, or weights of a neural network). The trained algorithm may be adjusted or tuned continuously during the training process or after the training process has completed.

After the trained algorithm is initially trained, a subset of the inputs may be identified as most influential or most important to be included for making high-quality classifications. For example, a subset of the plurality of pregnancy-related genomic loci may be identified as most influential or most important to be included for making high-quality classifications or identifications of pregnancy-related states (or sub-types of pregnancy-related states). The plurality of pregnancy-related genomic loci or a subset thereof may be ranked based on classification metrics indicative of each genomic locus's influence or importance toward making high-quality classifications or identifications of pregnancy-related states (or sub-types of pregnancy-related states). Such metrics may be used to reduce, in some cases significantly, the number of input variables (e.g., predictor variables) that may be used to train the trained algorithm to a desired performance level (e.g., based on a desired minimum accuracy, PPV, NPV, clinical sensitivity, clinical specificity, AUC, or a combination thereof).

For example, if training the trained algorithm with a plurality comprising several dozen or hundreds of input variables in the trained algorithm results in an accuracy of classification of more than 99%, then training the trained algorithm instead with only a selected subset of no more than about 5, no more than about 10, no more than about 15, no more than about 20, no more than about no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics.

### Identifying or Monitoring Pregnancy-Related States

After using a trained algorithm to process the dataset, the pregnancy-related state or pregnancy-related complication may be identified or monitored in the subject. The identification may be based at least in part on quantitative measures of sequence reads of the dataset at a panel of pregnancy-related genomic loci (e.g., quantitative measures of RNA transcripts or DNA at the pregnancy-related genomic loci), proteomic data comprising quantitative measures of proteins of the dataset at a panel of pregnancy-related proteins, and/or metabolome data comprising quantitative measures of a panel of pregnancy-related metabolites.

The pregnancy-related state may be identified in the subject at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The accuracy of identifying the pregnancy-related state by the trained algorithm may be calculated as the percentage of independent test samples (e.g., subjects known to have the pregnancy-related state or subjects with negative clinical test results for the pregnancy-related state) that are correctly identified or classified as having or not having the pregnancy-related state.

The pregnancy-related state may be identified in the subject with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the pregnancy-related state using the trained algorithm may be calculated as the percentage of biological samples identified or classified as having the pregnancy-related state that correspond to subjects that truly have the pregnancy-related state.

The pregnancy-related state may be identified in the subject with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the pregnancy-related state using the trained algorithm may be calculated as the percentage of biological samples identified or classified as not having the pregnancy-related state that correspond to subjects that truly do not have the pregnancy-related state.

The pregnancy-related state may be identified in the subject with a clinical sensitivity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the pregnancy-related state using the trained algorithm may be calculated as the percentage of independent test samples associated with presence of the pregnancy-related state (e.g., subjects known to have the pregnancy-related state) that are correctly identified or classified as having the pregnancy-related state.

The pregnancy-related state may be identified in the subject with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the pregnancy-related state using the trained algorithm may be calculated as the percentage of independent test samples associated with absence of the pregnancy-related state (e.g., subjects with negative clinical test results for the pregnancy-related state) that are correctly identified or classified as not having the pregnancy-related state.

The pregnancy-related state may be identified in the subject with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more.

Upon identifying the subject as having the pregnancy-related state, the subject may be optionally provided with a therapeutic intervention (e.g., prescribing an appropriate course of treatment to treat the pregnancy-related state of the subject). The therapeutic intervention may comprise a prescription of an effective dose of a drug, a further testing or evaluation of the pregnancy-related state, a further monitoring of the pregnancy-related state, an induction or inhibition of labor, or a combination thereof. If the subject is currently being treated for the pregnancy-related state with a course of treatment, the therapeutic intervention may comprise a subsequent different course of treatment (e.g., to increase treatment efficacy due to non-efficacy of the current course of treatment).

The therapeutic intervention may comprise recommending the subject for a secondary clinical test to confirm a diagnosis of the pregnancy-related state. This secondary clinical test may comprise an imaging test, a blood test, a computed tomography (CT) scan, a magnetic resonance imaging (MM) scan, an ultrasound scan, a chest X-ray, a positron emission tomography (PET) scan, a PET-CT scan, a biological cytology, an amniocentesis, a non-invasive prenatal test (NIPT), or any combination thereof.

The quantitative measures of sequence reads of the dataset at the panel of pregnancy-related genomic loci (e.g., quantitative measures of RNA transcripts or DNA at the pregnancy-related genomic loci), proteomic data comprising quantitative measures of proteins of the dataset at a panel of pregnancy-related proteins, and/or metabolome data comprising quantitative measures of a panel of pregnancy-related metabolites may be assessed over a duration of time to monitor a patient (e.g., subject who has pregnancy-related state or who is being treated for pregnancy-related state). In such cases, the quantitative measures of the dataset of the patient may change during the course of treatment. For example, the quantitative measures of the dataset of a patient with decreasing risk of the pregnancy-related state due to an effective treatment may shift toward the profile or distribution of a healthy subject (e.g., a subject without a pregnancy-related complication). Conversely, for example, the quantitative measures of the dataset of a patient with increasing risk of the pregnancy-related state due to an ineffective treatment may shift toward the profile or distribution of a subject with higher risk of the pregnancy-related state or a more advanced pregnancy-related state.

The pregnancy-related state of the subject may be monitored by monitoring a course of treatment for treating the pregnancy-related state of the subject. The monitoring may comprise assessing the pregnancy-related state of the subject at two or more time points. The assessing may be based at least on the quantitative measures of sequence reads of the dataset at a panel of pregnancy-related genomic loci (e.g., quantitative measures of RNA transcripts or DNA at the pregnancy-related genomic loci), proteomic data comprising quantitative measures of proteins of the dataset at a panel of pregnancy-related proteins, and/or metabolome data comprising quantitative measures of a panel of pregnancy-related metabolites determined at each of the two or more time points.

In some embodiments, a difference in the quantitative measures of sequence reads of the dataset at a panel of pregnancy-related genomic loci (e.g., quantitative measures of RNA transcripts or DNA at the pregnancy-related genomic loci), proteomic data comprising quantitative measures of proteins of the dataset at a panel of pregnancy-related proteins, and/or metabolome data comprising quantitative measures of a panel of pregnancy-related metabolites determined between the two or more time points may be indicative of one or more clinical indications, such as (i) a diagnosis of the pregnancy-related state of the subject, (ii) a prognosis of the pregnancy-related state of the subject, (iii) an increased risk of the pregnancy-related state of the subject, (iv) a decreased risk of the pregnancy-related state of the subject, (v) an efficacy of the course of treatment for treating the pregnancy-related state of the subject, and (vi) a non-efficacy of the course of treatment for treating the pregnancy-related state of the subject.

In some embodiments, a difference in the quantitative measures of sequence reads of the dataset at a panel of pregnancy-related genomic loci (e.g., quantitative measures of RNA transcripts or DNA at the pregnancy-related genomic loci), proteomic data comprising quantitative measures of proteins of the dataset at a panel of pregnancy-related proteins, and/or metabolome data comprising quantitative measures of a panel of pregnancy-related metabolites determined between the two or more time points may be indicative of a diagnosis of the pregnancy-related state of the subject. For example, if the pregnancy-related state was not detected in the subject at an earlier time point but was detected in the subject at a later time point, then the difference may be indicative of a diagnosis of the pregnancy-related state of the subject. A clinical action or decision may be made based on this indication of diagnosis of the pregnancy-related state of the subject, such as, for example, prescribing a new therapeutic intervention for the subject. The clinical action or decision may comprise recommending the subject for a secondary clinical test to confirm the diagnosis of the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a biological cytology, an amniocentesis, a non-invasive prenatal test (NIPT), or any combination thereof.

In some embodiments, a difference in the quantitative measures of sequence reads of the dataset at a panel of pregnancy-related genomic loci (e.g., quantitative measures of RNA transcripts or DNA at the pregnancy-related genomic loci), proteomic data comprising quantitative measures of proteins of the dataset at a panel of pregnancy-related proteins, and/or metabolome data comprising quantitative measures of a panel of pregnancy-related metabolites determined between the two or more time points may be indicative of a prognosis of the pregnancy-related state of the subject.

In some embodiments, a difference in the quantitative measures of sequence reads of the dataset at a panel of pregnancy-related genomic loci (e.g., quantitative measures of RNA transcripts or DNA at the pregnancy-related genomic loci), proteomic data comprising quantitative measures of proteins of the dataset at a panel of pregnancy-related proteins, and/or metabolome data comprising quantitative measures of a panel of pregnancy-related metabolites determined between the two or more time points may be indicative of the subject having an increased risk of the pregnancy-related state. For example, if the pregnancy-related state was detected in the subject both at an earlier time point and at a later time point, and if the difference is a positive difference (e.g., the quantitative measures of sequence reads of the dataset at a panel of pregnancy-related genomic loci (e.g., quantitative measures of RNA transcripts or DNA at the pregnancy-related genomic loci), proteomic data comprising quantitative measures of proteins of the dataset at a panel of pregnancy-related proteins, and/or metabolome data comprising quantitative measures of a panel of pregnancy-related metabolites increased from the earlier time point to the later time point), then the difference may be indicative of the subject having an increased risk of the pregnancy-related state. A clinical action or decision may be made based on this indication of the increased risk of the pregnancy-related state, e.g., prescribing a new therapeutic intervention or switching therapeutic interventions (e.g., ending a current treatment and prescribing a new treatment) for the subject. The clinical action or decision may comprise recommending the subject for a secondary clinical test to confirm the increased risk of the pregnancy-related state. This secondary clinical test may comprise an imaging test, a blood test, a computed tomography (CT) scan, a magnetic resonance imaging (MM) scan, an ultrasound scan, a chest X-ray, a positron emission tomography (PET) scan, a PET-CT scan, a biological cytology, an amniocentesis, a non-invasive prenatal test (NIPT), or any combination thereof.

In some embodiments, a difference in the quantitative measures of sequence reads of the dataset at a panel of pregnancy-related genomic loci (e.g., quantitative measures of RNA transcripts or DNA at the pregnancy-related genomic loci), proteomic data comprising quantitative measures of proteins of the dataset at a panel of pregnancy-related proteins, and/or metabolome data comprising quantitative measures of a panel of pregnancy-related metabolites determined between the two or more time points may be indicative of the subject having a decreased risk of the pregnancy-related state. For example, if the pregnancy-related state was detected in the subject both at an earlier time point and at a later time point, and if the difference is a negative difference (e.g., the quantitative measures of sequence reads of the dataset at a panel of pregnancy-related genomic loci (e.g., quantitative measures of RNA transcripts or DNA at the pregnancy-related genomic loci), proteomic data comprising quantitative measures of proteins of the dataset at a panel of pregnancy-related proteins, and/or metabolome data comprising quantitative measures of a panel of pregnancy-related metabolites decreased from the earlier time point to the later time point), then the difference may be indicative of the subject having a decreased risk of the pregnancy-related state. A clinical action or decision may be made based on this indication of the decreased risk of the pregnancy-related state (e.g., continuing or ending a current therapeutic intervention) for the subject. The clinical action or decision may comprise recommending the subject for a secondary clinical test to confirm the decreased risk of the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a biological cytology, an amniocentesis, a non-invasive prenatal test (NIPT), or any combination thereof.

In some embodiments, a difference in the quantitative measures of sequence reads of the dataset at a panel of pregnancy-related genomic loci (e.g., quantitative measures of RNA transcripts or DNA at the pregnancy-related genomic loci), proteomic data comprising quantitative measures of proteins of the dataset at a panel of pregnancy-related proteins, and/or metabolome data comprising quantitative measures of a panel of pregnancy-related metabolites determined between the two or more time points may be indicative of an efficacy of the course of treatment for treating the pregnancy-related state of the subject. For example, if the pregnancy-related state was detected in the subject at an earlier time point but was not detected in the subject at a later time point, then the difference may be indicative of an efficacy of the course of treatment for treating the pregnancy-related state of the subject. A clinical action or decision may be made based on this indication of the efficacy of the course of treatment for treating the pregnancy-related state of the subject, e.g., continuing or ending a current therapeutic intervention for the subject. The clinical action or decision may comprise recommending the subject for a secondary clinical test to confirm the efficacy of the course of treatment for treating the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a biological cytology, an amniocentesis, a non-invasive prenatal test (NIPT), or any combination thereof.

In some embodiments, a difference in the quantitative measures of sequence reads of the dataset at a panel of pregnancy-related genomic loci (e.g., quantitative measures of RNA transcripts or DNA at the pregnancy-related genomic loci), proteomic data comprising quantitative measures of proteins of the dataset at a panel of pregnancy-related proteins, and/or metabolome data comprising quantitative measures of a panel of pregnancy-related metabolites determined between the two or more time points may be indicative of a non-efficacy of the course of treatment for treating the pregnancy-related state of the subject. For example, if the pregnancy-related state was detected in the subject both at an earlier time point and at a later time point, and if the difference is a positive or zero difference (e.g., the quantitative measures of sequence reads of the dataset at a panel of pregnancy-related genomic loci (e.g., quantitative measures of RNA transcripts or DNA at the pregnancy-related genomic loci), proteomic data comprising quantitative measures of proteins of the dataset at a panel of pregnancy-related proteins, and/or metabolome data comprising quantitative measures of a panel of pregnancy-related metabolites increased or remained at a constant level from the earlier time point to the later time point), and if an efficacious treatment was indicated at an earlier time point, then the difference may be indicative of a non-efficacy of the course of treatment for treating the pregnancy-related state of the subject. A clinical action or decision may be made based on this indication of the non-efficacy of the course of treatment for treating the pregnancy-related state of the subject, e.g., ending a current therapeutic intervention and/or switching to (e.g., prescribing) a different new therapeutic intervention for the subject. The clinical action or decision may comprise recommending the subject for a secondary clinical test to confirm the non-efficacy of the course of treatment for treating the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a biological cytology, an amniocentesis, a non-invasive prenatal test (NIPT), or any combination thereof.

After the pregnancy-related state is identified or an increased risk of the pregnancy-related state is monitored in the subject, a report may be electronically outputted that is indicative of (e.g., identifies or provides an indication of) the pregnancy-related state of the subject. The subject may not display a pregnancy-related state (e.g., is asymptomatic of the pregnancy-related state such as a pregnancy-related complication). The report may be presented on a graphical user interface (GUI) of an electronic device of a user. The user may be the subject, a caretaker, a physician, a nurse, or another health care worker.

The report may include one or more clinical indications such as (i) a diagnosis of the pregnancy-related state of the subject, (ii) a prognosis of the pregnancy-related state of the subject, (iii) an increased risk of the pregnancy-related state of the subject, (iv) a decreased risk of the pregnancy-related state of the subject, (v) an efficacy of the course of treatment for treating the pregnancy-related state of the subject, and (vi) a non-efficacy of the course of treatment for treating the pregnancy-related state of the subject. The report may include one or more clinical actions or decisions made based on these one or more clinical indications. Such clinical actions or decisions may be directed to therapeutic interventions, induction or inhibition of labor, or further clinical assessment or testing of the pregnancy-related state of the subject.

For example, a clinical indication of a diagnosis of the pregnancy-related state of the subject may be accompanied with a clinical action of prescribing a new therapeutic intervention for the subject. As another example, a clinical indication of an increased risk of the pregnancy-related state of the subject may be accompanied with a clinical action of prescribing a new therapeutic intervention or switching therapeutic interventions (e.g., ending a current treatment and prescribing a new treatment) for the subject. As another example, a clinical indication of a decreased risk of the pregnancy-related state of the subject may be accompanied with a clinical action of continuing or ending a current therapeutic intervention for the subject. As another example, a clinical indication of an efficacy of the course of treatment for treating the pregnancy-related state of the subject may be accompanied with a clinical action of continuing or ending a current therapeutic intervention for the subject. As another example, a clinical indication of a non-efficacy of the course of treatment for treating the pregnancy-related state of the subject may be accompanied with a clinical action of ending a current therapeutic intervention and/or switching to (e.g., prescribing) a different new therapeutic intervention for the subject.

### Computer Systems

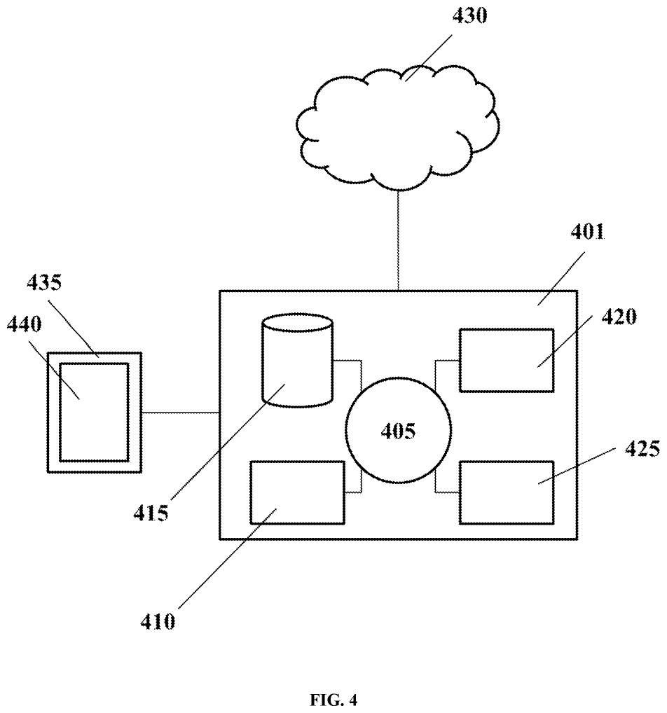

The present disclosure provides computer systems that are programmed to implement methods of the disclosure. FIG. 4 shows a computer system 401 that is programmed or otherwise configured to, for example, (i) evaluate a subject for participation in a clinical trial, (ii) select a subject for a clinical trial, (iii) direct collection of a biological sample of the subject, (iv) train and test a trained algorithm, (v) use the trained algorithm to process data to determine a pregnancy-related state of a subject, (vi) determine a quantitative measure indicative of a pregnancy-related state of a subject, (vii) identify or monitor the pregnancy-related state of the subject, and (viii) electronically output a report that indicative of the pregnancy-related state of the subject.

The computer system 401 can regulate various aspects of analysis, calculation, and generation of the present disclosure, such as, for example, (i) evaluating a subject for participation in a clinical trial, (ii) selecting a subject for a clinical trial, (iii) directing collection of a biological sample of the subject, (iv) training and testing a trained algorithm, (v) using the trained algorithm to process data to determine a pregnancy-related state of a subject, (vi) determining a quantitative measure indicative of a pregnancy-related state of a subject, (vii) identifying or monitoring the pregnancy-related state of the subject, and (viii) electronically outputting a report that indicative of the pregnancy-related state of the subject. The computer system 401 can be an electronic device of a user or a computer system that is remotely located with respect to the electronic device. The electronic device can be a mobile electronic device.

The computer system 401 includes a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk), communication interface 420 (e.g., network adapter) for communicating with one or more other systems, and peripheral devices 425, such as cache, other memory, data storage and/or electronic display adapters. The memory 410, storage unit 415, interface 420 and peripheral devices 425 are in communication with the CPU 405 through a communication bus (solid lines), such as a motherboard. The storage unit 415 can be a data storage unit (or data repository) for storing data. The computer system 401 can be operatively coupled to a computer network (“network”) 430 with the aid of the communication interface 420. The network 430 can be the Internet, an internet and/or extranet, or an intranet and/or extranet that is in communication with the Internet.

The network 430 in some cases is a telecommunication and/or data network. The network 430 can include one or more computer servers, which can enable distributed computing, such as cloud computing. For example, one or more computer servers may enable cloud computing over the network 430 (“the cloud”) to perform various aspects of analysis, calculation, and generation of the present disclosure, such as, for example, (i) evaluating a subject for participation in a clinical trial, (ii) selecting a subject for a clinical trial, (iii) directing collection of a biological sample of the subject, (iv) training and testing a trained algorithm, (v) using the trained algorithm to process data to determine a pregnancy-related state of a subject, (vi) determining a quantitative measure indicative of a pregnancy-related state of a subject, (vii) identifying or monitoring the pregnancy-related state of the subject, and (viii) electronically outputting a report that indicative of the pregnancy-related state of the subject. Such cloud computing may be provided by cloud computing platforms such as, for example, Amazon Web Services (AWS), Microsoft Azure, Google Cloud Platform, and IBM cloud. The network 430, in some cases with the aid of the computer system 401, can implement a peer-to-peer network, which may enable devices coupled to the computer system 401 to behave as a client or a server.

The CPU 405 may comprise one or more computer processors and/or one or more graphics processing units (GPUs). The CPU 405 can execute a sequence of machine-readable instructions, which can be embodied in a program or software. The instructions may be stored in a memory location, such as the memory 410. The instructions can be directed to the CPU 405, which can subsequently program or otherwise configure the CPU 405 to implement methods of the present disclosure. Examples of operations performed by the CPU 405 can include fetch, decode, execute, and writeback.

The CPU 405 can be part of a circuit, such as an integrated circuit. One or more other components of the system 401 can be included in the circuit. In some cases, the circuit is an application specific integrated circuit (ASIC).

The storage unit 415 can store files, such as drivers, libraries and saved programs. The storage unit 415 can store user data, e.g., user preferences and user programs. The computer system 401 in some cases can include one or more additional data storage units that are external to the computer system 401, such as located on a remote server that is in communication with the computer system 401 through an intranet or the Internet.

The computer system 401 can communicate with one or more remote computer systems through the network 430. For instance, the computer system 401 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 401 via the network 430.

Methods as described herein can be implemented by way of machine (e.g., computer processor) executable code stored on an electronic storage location of the computer system 401, such as, for example, on the memory 410 or electronic storage unit 415. The machine executable or machine-readable code can be provided in the form of software. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 401 can include or be in communication with an electronic display 435 that comprises a user interface (UI) 440 for providing, for example, (i) advertisements of clinical trials to a subject, (ii) recruitment web pages for the clinical trial, (iii) an eligibility questionnaires or eligibility criteria for the clinical trial, (iv) a visual display indicative of recruiting, evaluating, or selecting a subject for a clinical trial, (v) a visual display indicative of training and testing of a trained algorithm, (vi) a visual display of data indicative of a pregnancy-related state of a subject, (vii) a quantitative measure of a pregnancy-related state of a subject, (viii) an identification of a subject as having a pregnancy-related state, or (ix) an electronic report indicative of the pregnancy-related state of the subject. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 405. The algorithm can, for example, (i) evaluate a subject for participation in a clinical trial, (ii) select a subject for a clinical trial, (iii) direct collection of a biological sample of the subject, (iv) train and test a trained algorithm, (v) use the trained algorithm to process data to determine a pregnancy-related state of a subject, (vi) determine a quantitative measure indicative of a pregnancy-related state of a subject, (vii) identify or monitor the pregnancy-related state of the subject, and (viii) electronically output a report that indicative of the pregnancy-related state of the subject.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Embodiments

The following non-limiting embodiments provide illustrative examples of the invention, but do not limit the scope of the invention.
Embodiment 1. A method for directing a clinical trial, comprising:
(a) evaluating a subject for participation in the clinical trial, wherein the clinical trial is for assessing pregnancy-related states of subjects or fetuses thereof;
(b) selecting the subject for the clinical trial, based at least in part on the evaluating in (a); and
(c) directing collection of a biological sample of the subject at a location remote with respect to a health care facility.
Embodiment 2. The method of embodiment 1, wherein the subject is pregnant or is suspected of being pregnant.
Embodiment 3. The method of embodiment 1 or 2, wherein the pregnancy-related states of the subjects or the fetuses thereof comprise prenatal pregnancy-related states of the subjects or the fetuses thereof.
Embodiment 4. The method of any one of embodiments 1-3, wherein the pregnancy-related states of the subjects or the fetuses thereof comprise postpartum or postnatal pregnancy-related states of the subjects or the fetuses thereof.
Embodiment 5. The method of embodiment 4, wherein the postpartum or postnatal pregnancy-related states are selected from the group consisting of cardiovascular diseases, depression, anxiety, post GDM, post-partum complications of preeclampsia, excessive bleeding after giving birth, pulmonary embolism, cardiomyopathy, diabetes, anemia, and hypertensive disorders.
Embodiment 6. The method of any one of embodiments 1-5, wherein the pregnancy-related states are selected from the group consisting of pre-term birth, full-term birth, gestational age, due date, onset of labor, a pregnancy-related hypertensive disorder, preeclampsia, eclampsia, gestational diabetes, a congenital disorder of a fetus of the subject, ectopic pregnancy, spontaneous abortion, stillbirth, a post-partum complication, hyperemesis gravidarum, hemorrhage or excessive bleeding during delivery, premature rupture of membrane, premature rupture of membrane in pre-term birth, placenta previa, intrauterine fetal growth restriction, macrosomia, a neonatal condition, a fertility-related condition, and a fetal development stage or state.
Embodiment 7. The method of any one of embodiments 1-6, wherein the pregnancy-related states comprise the pre-term birth.
Embodiment 8. The method of any one of embodiments 1-6, wherein the pregnancy-related states comprise the pre-eclampsia.
Embodiment 9. The method of any one of embodiments 1-6, wherein the pregnancy-related states comprise the due date.
Embodiment 10. The method of any one of embodiments 1-9, further comprising recruiting the subject for participation in the clinical trial.
Embodiment 11. The method of embodiment 10, wherein the recruiting comprises use of a digital marketing campaign.
Embodiment 12. The method of embodiment 10 or 11, wherein the recruiting comprises displaying advertisements to the subject through a computer network.
Embodiment 13. The method of embodiment 12, wherein the advertisements are displayed through a mobile device of the subject.
Embodiment 14. The method of embodiment 13, wherein the mobile device comprises a smartphone, a laptop, a tablet computer, a smartwatch, or a combination thereof
Embodiment 15. The method of embodiment 12, wherein the advertisements are displayed or viewed through a social media channel, social networking, pregnancy education, pregnancy tracker, menstruation tracker, ovulation tracker, or fertility tracker platform.
Embodiment 16. The method of embodiment 12, wherein recruiting the subject comprises, responsive to the subject responding to the displayed advertisement, directing the subject to visit or view one or more web pages of a user experience (UX) journey.
Embodiment 17. The method of embodiment 16, wherein the subject responds to the displayed advertisement by clicking a hyperlink of the displayed advertisement.
Embodiment 18. The method of embodiment 16, wherein recruiting the subject comprises displaying a contact form to the subject for completing, and receiving the completed contact form comprising personal contact information from the subject.
Embodiment 19. The method of embodiment 16, further comprising displaying an eligibility questionnaire or a set of eligibility criteria to the subject via the one or more web pages.
Embodiment 20. The method of embodiment 19, further comprising collecting response data from the subject using the eligibility questionnaire, a self-reported family history survey, a demographic survey, a medical condition survey, a customer satisfaction survey, or a combination thereof.
Embodiment 21. The method of embodiment 20, wherein evaluating the subject for participation in the clinical trial comprises processing the response data collected from the subject.
Embodiment 22. The method of embodiment 21, wherein processing the response data collected from the subject comprises determining a gestational age of the fetus of the subject and/or a time window for the collection of the biological sample of the subject.
Embodiment 23. The method of any one of embodiments 1-22, wherein evaluating the subject for participation in the clinical trial comprises applying an inclusion criterion or an exclusion criterion of the clinical trial.
Embodiment 24. The method of embodiment 23, wherein the inclusion criterion or the exclusion criterion is related to age, race, ethnicity, body mass index, gestational age, or location of the subject or a fetus of the subject.
Embodiment 25. The method of embodiment 23, further comprising selecting the subject for the clinical trial when the subject satisfies the inclusion criterion.
Embodiment 26. The method of embodiment 25, further comprising obtaining informed consent from the subject.
Embodiment 27. The method of embodiment 26, wherein the informed consent is obtained virtually using electronic signatures.
Embodiment 28. The method of embodiment 26, wherein the informed consent is obtained for research use of the biological sample of the subject and data resulting therefrom.
Embodiment 29. The method of embodiment 26, wherein the informed consent is obtained for access to at least a portion of medical records of the subject or the fetus thereof
Embodiment 30. The method of embodiment 29, wherein the informed consent comprises authorization for a health care provider to release the medical records of the subject or the fetus thereof.
Embodiment 31. The method of embodiment 29 or 30, wherein the medical records comprise one or more members selected from the group consisting of obstetric ultrasound scans, Doppler ultrasound scan of blood flow, cervical length, neonatal intensive care unit (NICU) records, hospital delivery notes, blood test results, newborn evaluation immediately following birth, Apgar score of a newborn, records of scheduled prenatal visits, and records of postpartum or postnatal checkups.
Embodiment 32. The method of embodiment 23, further comprising excluding the subject for the clinical trial when the subject satisfies the exclusion criterion.
Embodiment 33. The method of any one of embodiments 1-32, wherein the biological sample is selected from the group consisting of a blood sample, a urine sample, a stool sample, a saliva sample, a vaginal sample, a cervical sample, and a swab sample.
Embodiment 34. The method of embodiment 33, wherein the biological sample comprises the blood sample.
Embodiment 35. The method of embodiment 34, wherein (c) comprises shipping a biological sample collection kit to the location, and receiving a return shipment of the collected biological sample from the location.
Embodiment 36. The method of embodiment 34, wherein the blood sample is obtained from the subject by a phlebotomy service.
Embodiment 37. The method of embodiment 36, wherein the phlebotomy service is a mobile phlebotomy service.
Embodiment 38. The method of embodiment 36, wherein (c) comprises shipping a biological sample collection kit to a phlebotomist, and receiving a return shipment of the collected biological sample from the phlebotomist.
Embodiment 39. The method of any one of embodiments 1-38, wherein the location is a residence, a workplace, or another location of choice of the subject.
Embodiment 40. The method of any one of embodiments 1-39, wherein the clinical trial is an interventional clinical trial.
Embodiment 41. The method of embodiment 40, wherein the interventional clinical trial is directed at least in part through a telemedicine consultation with a health care provider of the subject.
Embodiment 42. The method of any one of embodiments 1-39, wherein the clinical trial is an observational clinical trial.
Embodiment 43. The method of any one of embodiments 1-42, wherein the clinical trial is a longitudinal clinical trial.
Embodiment 44. The method of any one of embodiments 1-43, wherein the clinical trial is an Institutional Review Board (IRB) approved clinical trial.
Embodiment 45. A method for directing a clinical trial, comprising:
(a) evaluating a subject for participation in the clinical trial, wherein the clinical trial is for assessing pregnancy-related states of fetuses of subjects; and
(b) selecting the subject for the clinical trial, based at least in part on the evaluating in (a), wherein the clinical trial comprises collection of data in absence of the subject visiting a health care facility.
Embodiment 46. The method of embodiment 45, wherein the subject is pregnant or is suspected of being pregnant.
Embodiment 47. The method of embodiment 45 or 46, wherein the pregnancy-related states of the fetuses of the subjects comprise prenatal pregnancy-related states of the fetuses of the subjects.
Embodiment 48. The method of any one of embodiments 45-47, wherein the pregnancy-related states of the fetuses of the subjects comprise postpartum or postnatal pregnancy-related states of the fetuses of the subjects.
Embodiment 49. The method of embodiment 48, wherein the postpartum or postnatal pregnancy-related states are selected from the group consisting of cardiovascular diseases, depression, anxiety, post GDM, post-partum complications of preeclampsia, excessive bleeding after giving birth, pulmonary embolism, cardiomyopathy, diabetes, anemia, and hypertensive disorders.
Embodiment 50. The method of any one of embodiments 45-49, wherein the pregnancy-related states are selected from the group consisting of pre-term birth, full-term birth, gestational age, due date, onset of labor, a congenital disorder of the fetus of the subject, ectopic pregnancy, spontaneous abortion, stillbirth, intrauterine fetal growth restriction, macrosomia, a neonatal condition, and a fetal development stage or state.
Embodiment 51. The method of embodiment 50, wherein the pregnancy-related states comprise the pre-term birth.
Embodiment 52. The method of embodiment 50, wherein the pregnancy-related states comprise the due date.
Embodiment 53. The method of any one of embodiments 45-52, further comprising recruiting the subject for participation in the clinical trial.
Embodiment 54. The method of embodiment 53, wherein the recruiting comprises use of a digital marketing campaign.
Embodiment 55. The method of embodiment 53 or 54, wherein the recruiting comprises displaying advertisements to the subject through a computer network.
Embodiment 56. The method of embodiment 55, wherein the advertisements are displayed through a mobile device of the subject.
Embodiment 57. The method of embodiment 56, wherein the mobile device comprises a smartphone, a laptop, a tablet computer, a smartwatch, or a combination thereof
Embodiment 58. The method of embodiment 55, wherein the advertisements are displayed or viewed through a social media channel, social networking, pregnancy education, pregnancy tracker, menstruation tracker, ovulation tracker, or fertility tracker platform.
Embodiment 59. The method of embodiment 55, wherein recruiting the subject comprises, responsive to the subject responding to the displayed advertisement, directing the subject to visit or view one or more web pages of a user experience (UX) journey.
Embodiment 60. The method of embodiment 59, wherein the subject responds to the displayed advertisement by clicking a hyperlink of the displayed advertisement.
Embodiment 61. The method of embodiment 59, wherein recruiting the subject comprises displaying a contact form to the subject for completing, and receiving the completed contact form comprising personal contact information from the subject.
Embodiment 62. The method of embodiment 59, further comprising displaying an eligibility questionnaire or a set of eligibility criteria to the subject via the one or more web pages.
Embodiment 63. The method of embodiment 62, further comprising collecting response data from the subject using the eligibility questionnaire, a self-reported family history survey, a demographic survey, a medical condition survey, a customer satisfaction survey, or a combination thereof.
Embodiment 64. The method of embodiment 63, wherein evaluating the subject for participation in the clinical trial comprises processing the response data collected from the subject.
Embodiment 65. The method of embodiment 64, wherein processing the response data collected from the subject comprises determining a gestational age of the fetus of the subject and/or a time window for the collection of the biological sample of the subject.
Embodiment 66. The method of any one of embodiments 45-65, wherein evaluating the subject for participation in the clinical trial comprises applying an inclusion criterion or an exclusion criterion of the clinical trial.
Embodiment 67. The method of embodiment 66, wherein the inclusion criterion or the exclusion criterion is related to age, race, ethnicity, body mass index, gestational age, or location of the subject or a fetus of the subject.
Embodiment 68. The method of embodiment 66, further comprising selecting the subject for the clinical trial when the subject satisfies the inclusion criterion.
Embodiment 69. The method of embodiment 68, further comprising obtaining informed consent from the subject.
Embodiment 70. The method of embodiment 69, wherein the informed consent is obtained virtually using electronic signatures.
Embodiment 71. The method of embodiment 69, wherein the informed consent is obtained for research use of the biological sample of the subject and data resulting therefrom.
Embodiment 72. The method of embodiment 69, wherein the informed consent is obtained for access to at least a portion of medical records of the subject or the fetus thereof
Embodiment 73. The method of embodiment 72, wherein the informed consent comprises authorization for a health care provider to release the medical records of the subject or the fetus thereof.
Embodiment 74. The method of embodiment 72 or 73, wherein the medical records comprise one or more members selected from the group consisting of obstetric ultrasound scans, Doppler ultrasound scan of blood flow, cervical length, neonatal intensive care unit (NICU) records, hospital delivery notes, blood test results, newborn evaluation immediately following birth, Apgar score of a newborn, records of scheduled prenatal visits, and records of postpartum or postnatal checkups.
Embodiment 75. The method of embodiment 66, further comprising excluding the subject for the clinical trial when the subject satisfies the exclusion criterion.
Embodiment 76. The method of any one of embodiments 45-75, wherein the collection of data comprises assaying a biological sample of the subject.
Embodiment 77. The method of embodiment 76, wherein the biological sample is selected from the group consisting of a blood sample, a urine sample, a stool sample, a saliva sample, a vaginal sample, a cervical sample, and a swab sample.
Embodiment 78. The method of embodiment 76, wherein the biological sample comprises the blood sample.
Embodiment 79. The method of embodiment 76, wherein the collection of data comprises shipping a biological sample collection kit to the location, and receiving a return shipment of the collected biological sample from the location.
Embodiment 80. The method of embodiment 78, wherein the blood sample is obtained from the subject by a phlebotomy service.
Embodiment 81. The method of embodiment 80, wherein the phlebotomy service is a mobile phlebotomy service.
Embodiment 82. The method of embodiment 80, wherein the collection of data comprises shipping a biological sample collection kit to a phlebotomist, and receiving a return shipment of the collected biological sample from the phlebotomist.
Embodiment 83. The method of any one of embodiments 45-82, wherein the biological sample is obtained from the subject at a location remote with respect to a health care facility.
Embodiment 84. The method of embodiment 83, wherein the location is a residence, a workplace, or another location of choice of the subject.
Embodiment 85. The method of any one of embodiments 45-84, wherein the clinical trial is an interventional clinical trial.
Embodiment 86. The method of embodiment 85, wherein the interventional clinical trial is directed at least in part through a telemedicine consultation with a health care provider of the subject.
Embodiment 87. The method of any one of embodiments 45-84, wherein the clinical trial is an observational clinical trial.
Embodiment 88. The method of any one of embodiments 45-87, wherein the clinical trial is a longitudinal clinical trial.
Embodiment 89. The method of any one of embodiments 45-88, wherein the clinical trial is an Institutional Review Board (IRB) approved clinical trial.
Embodiment 90. A method for directing a clinical trial, comprising:
(a) evaluating a subject for participation in the clinical trial, wherein the clinical trial is for assessing pregnancy-related states of subjects or fetuses thereof, wherein the subjects are in a first trimester or a second trimester of pregnancy; and
(b) selecting the subject for the clinical trial, based at least in part on the evaluating in (a), wherein the clinical trial comprises collection of data in absence of the subject visiting a health care facility.
Embodiment 91. The method of embodiment 90, wherein the subject is pregnant or is suspected of being pregnant.
Embodiment 92. The method of embodiment 90 or 91, wherein the pregnancy-related states of the subjects or the fetuses thereof comprise prenatal pregnancy-related states of the subjects or the fetuses thereof.
Embodiment 93. The method of any one of embodiments 90-92, wherein the pregnancy-related states of the subjects or the fetuses thereof comprise postpartum or postnatal pregnancy-related states of the subjects or the fetuses thereof.
Embodiment 94. The method of embodiment 93, wherein the postpartum or postnatal pregnancy-related states are selected from the group consisting of cardiovascular diseases, depression, anxiety, post GDM, post-partum complications of preeclampsia, excessive bleeding after giving birth, pulmonary embolism, cardiomyopathy, diabetes, anemia, and hypertensive disorders.
Embodiment 95. The method of any one of embodiments 90-94, wherein the pregnancy-related states are selected from the group consisting of pre-term birth, full-term birth, gestational age, due date, onset of labor, a pregnancy-related hypertensive disorder, preeclampsia, eclampsia, gestational diabetes, a congenital disorder of a fetus of the subject, ectopic pregnancy, spontaneous abortion, stillbirth, a post-partum complication, hyperemesis gravidarum, hemorrhage or excessive bleeding during delivery, premature rupture of membrane, premature rupture of membrane in pre-term birth, placenta previa, intrauterine fetal growth restriction, macrosomia, a neonatal condition, a fertility-related condition, and a fetal development stage or state.
Embodiment 96. The method of embodiment 95, wherein the pregnancy-related states comprise the pre-term birth.
Embodiment 97. The method of embodiment 95, wherein the pregnancy-related states comprise the pre-eclampsia.
Embodiment 98. The method of embodiment 95, wherein the pregnancy-related states comprise the due date.
Embodiment 99. The method of any one of embodiments 90-98, further comprising recruiting the subject for participation in the clinical trial.
Embodiment 100. The method of embodiment 99, wherein the recruiting comprises use of a digital marketing campaign.
Embodiment 101. The method of embodiment 99 or 100, wherein the recruiting comprises displaying advertisements to the subject through a computer network.
Embodiment 102. The method of embodiment 101, wherein the advertisements are displayed through a mobile device of the subject.
Embodiment 103. The method of embodiment 102, wherein the mobile device comprises a smartphone, a laptop, a tablet computer, a smartwatch, or a combination thereof
Embodiment 104. The method of embodiment 101, wherein the advertisements are displayed or viewed through a social media channel, social networking, pregnancy education, pregnancy tracker, menstruation tracker, ovulation tracker, or fertility tracker platform.
Embodiment 105. The method of embodiment 101, wherein recruiting the subject comprises, responsive to the subject responding to the displayed advertisement, directing the subject to visit or view one or more web pages of a user experience (UX) journey.
Embodiment 106. The method of embodiment 105, wherein the subject responds to the displayed advertisement by clicking a hyperlink of the displayed advertisement.
Embodiment 107. The method of embodiment 105, wherein recruiting the subject comprises displaying a contact form to the subject for completing, and receiving the completed contact form comprising personal contact information from the subject.
Embodiment 108. The method of embodiment 105, further comprising displaying an eligibility questionnaire or a set of eligibility criteria to the subject via the one or more web pages.
Embodiment 109. The method of embodiment 108, further comprising collecting response data from the subject using the eligibility questionnaire, a self-reported family history survey, a demographic survey, a medical condition survey, a customer satisfaction survey, or a combination thereof.
Embodiment 110. The method of embodiment 109, wherein evaluating the subject for participation in the clinical trial comprises processing the response data collected from the subject.
Embodiment 111. The method of embodiment 110, wherein processing the response data collected from the subject comprises determining a gestational age of the fetus of the subject and/or a time window for the collection of the biological sample of the subject.
Embodiment 112. The method of embodiment 90, wherein evaluating the subject for participation in the clinical trial comprises applying an inclusion criterion or an exclusion criterion of the clinical trial.
Embodiment 113. The method of embodiment 112, wherein the inclusion criterion or the exclusion criterion is related to age, race, ethnicity, body mass index, gestational age, or location of the subject or a fetus of the subject.
Embodiment 114. The method of embodiment 112, further comprising selecting the subject for the clinical trial when the subject satisfies the inclusion criterion.
Embodiment 115. The method of embodiment 114, further comprising obtaining informed consent from the subject.
Embodiment 116. The method of embodiment 115, wherein the informed consent is obtained virtually using electronic signatures.
Embodiment 117. The method of embodiment 115, wherein the informed consent is obtained for research use of the biological sample of the subject and data resulting therefrom.
Embodiment 118. The method of embodiment 115, wherein the informed consent is obtained for access to at least a portion of medical records of the subject or the fetus thereof
Embodiment 119. The method of embodiment 118, wherein the informed consent comprises authorization for a health care provider to release the medical records of the subject or the fetus thereof.
Embodiment 120. The method of embodiment 118 or 119, wherein the medical records comprise one or more members selected from the group consisting of obstetric ultrasound scans, Doppler ultrasound scan of blood flow, cervical length, neonatal intensive care unit (NICU) records, hospital delivery notes, blood test results, newborn evaluation immediately following birth, Apgar score of a newborn, records of scheduled prenatal visits, and records of postpartum or postnatal checkups.
Embodiment 121. The method of embodiment 112, further comprising excluding the subject for the clinical trial when the subject satisfies the exclusion criterion.
Embodiment 122. The method of any one of embodiments 90-121, wherein the collection of data comprises assaying a biological sample of the subject.
Embodiment 123. The method of embodiment 122, wherein the biological sample is selected from the group consisting of a blood sample, a urine sample, a stool sample, a saliva sample, a vaginal sample, a cervical sample, and a swab sample.
Embodiment 124. The method of embodiment 122, wherein the biological sample comprises the blood sample.
Embodiment 125. The method of embodiment 122, wherein the collection of data comprises shipping a biological sample collection kit to the location, and receiving a return shipment of the collected biological sample from the location.
Embodiment 126. The method of embodiment 124, wherein the blood sample is obtained from the subject by a phlebotomy service.
Embodiment 127. The method of embodiment 126, wherein the phlebotomy service is a mobile phlebotomy service.
Embodiment 128. The method of embodiment 126, wherein the collection of data comprises shipping a biological sample collection kit to a phlebotomist, and receiving a return shipment of the collected biological sample from the phlebotomist.
Embodiment 129. The method of any one of embodiments 90-128, wherein the biological sample is obtained from the subject at a location remote with respect to a health care facility.
Embodiment 130. The method of embodiment 129, wherein the location is a residence, a workplace, or another location of choice of the subject.
Embodiment 131. The method of any one of embodiments 90-130, wherein the clinical trial is an interventional clinical trial.
Embodiment 132. The method of embodiment 131, wherein the interventional clinical trial is directed at least in part through a telemedicine consultation with a health care provider of the subject.
Embodiment 133. The method of any one of embodiments 90-130, wherein the clinical trial is an observational clinical trial.
Embodiment 134. The method of any one of embodiments 90-133, wherein the clinical trial is a longitudinal clinical trial.
Embodiment 135. The method of any one of embodiments 90-134, wherein the clinical trial is an Institutional Review Board (IRB) approved clinical trial.
Embodiment 136. A system comprising one or more computer processors that are individually or collectively programmed to implement a method for directing a clinical trial, the method comprising:
(a) evaluating a subject for participation in the clinical trial, wherein the clinical trial is for assessing pregnancy-related states of subjects or fetuses thereof;
(b) selecting the subject for the clinical trial, based at least in part on the evaluating in (a);
and
(c) directing collection of a biological sample of the subject at a location remote with respect to a health care facility.
Embodiment 137. A system comprising one or more computer processors that are individually or collectively programmed to implement a method for directing a clinical trial, the method comprising:
(a) evaluating a subject for participation in the clinical trial, wherein the clinical trial is for assessing pregnancy-related states of fetuses of subjects; and
(b) selecting the subject for the clinical trial, based at least in part on the evaluating in (a), wherein the clinical trial comprises collection of data in absence of the subject visiting a health care facility.
Embodiment 138. A system comprising one or more computer processors that are individually or collectively programmed to implement a method for directing a clinical trial, the method comprising:
(a) evaluating a subject for participation in the clinical trial, wherein the clinical trial is for assessing pregnancy-related states of subjects or fetuses thereof, wherein the subjects are in a first trimester or a second trimester of pregnancy; and
(b) selecting the subject for the clinical trial, based at least in part on the evaluating in (a), wherein the clinical trial comprises collection of data in absence of the subject visiting a health care facility.
Embodiment 139. A non-transitory computer-readable medium comprising machine-executable code that, upon execution by one or more computer processors, implements a method for directing a clinical trial, the method comprising:
(a) evaluating a subject for participation in the clinical trial, wherein the clinical trial is for assessing pregnancy-related states of subjects or fetuses thereof;
(b) selecting the subject for the clinical trial, based at least in part on the evaluating in (a); and
(c) directing collection of a biological sample of the subject at a location remote with respect to a health care facility.
Embodiment 140. A non-transitory computer-readable medium comprising machine-executable code that, upon execution by one or more computer processors, implements a method for directing a clinical trial, the method comprising:
(a) evaluating a subject for participation in the clinical trial, wherein the clinical trial is for assessing pregnancy-related states of fetuses of subjects; and
(b) selecting the subject for the clinical trial, based at least in part on the evaluating in (a), wherein the clinical trial comprises collection of data in absence of the subject visiting a health care facility.
Embodiment 141. A non-transitory computer-readable medium comprising machine-executable code that, upon execution by one or more computer processors, implements a method for directing a clinical trial, the method comprising:
(a) evaluating a subject for participation in the clinical trial, wherein the clinical trial is for assessing pregnancy-related states of subjects or fetuses thereof, wherein the subjects are in a first trimester or a second trimester of pregnancy; and
(b) selecting the subject for the clinical trial, based at least in part on the evaluating in (a),
wherein the clinical trial comprises collection of data in absence of the subject visiting a health care facility.

1.-95. (canceled)

96. A method for directing a clinical trial, comprising:
(a) evaluating a subject for participation in the clinical trial, wherein the clinical trial is for assessing pregnancy-related states of subjects or fetuses thereof;
(b) selecting the subject for the clinical trial, based at least in part on the evaluating in (a); and
(c) directing collection of a biological sample of the subject at a location remote with respect to a health care facility.

97. The method of claim 96, wherein the subject is pregnant or is suspected of being pregnant.

98. The method of claim 96, wherein the pregnancy-related states of the subjects or the fetuses thereof comprise prenatal pregnancy-related states of the subjects or the fetuses thereof.

99. The method of claim 96, wherein the pregnancy-related states of the subjects or the fetuses thereof comprise postpartum or postnatal pregnancy-related states of the subjects or the fetuses thereof.

100. The method of claim 99, wherein the postpartum or postnatal pregnancy-related states are selected from the group consisting of cardiovascular diseases, depression, anxiety, post GDM, post-partum complications of preeclampsia, excessive bleeding after giving birth, pulmonary embolism, cardiomyopathy, diabetes, anemia, and hypertensive disorders.

101. The method of claim 96, wherein the pregnancy-related states are selected from the group consisting of pre-term birth, full-term birth, gestational age, due date, onset of labor, a pregnancy-related hypertensive disorder, preeclampsia, eclampsia, gestational diabetes, a congenital disorder of a fetus of the subject, ectopic pregnancy, spontaneous abortion, stillbirth, a post-partum complication, hyperemesis gravidarum, hemorrhage or excessive bleeding during delivery, premature rupture of membrane, premature rupture of membrane in pre-term birth, placenta previa, intrauterine fetal growth restriction, macrosomia, a neonatal condition, a fertility-related condition, and a fetal development stage or state.

102. The method of claim 101, wherein the pregnancy-related states comprise the pre-term birth.

103. The method of claim 101, wherein the pregnancy-related states comprise the pre-eclampsia.

104. The method of claim 96, further comprising recruiting the subject for participation in the clinical trial.

105. The method of claim 104, wherein the recruiting comprises use of a digital marketing campaign.

106. The method of claim 104, wherein the recruiting comprises displaying advertisements to the subject through a computer network.

107. The method of claim 106, wherein the advertisements are displayed through a mobile device of the subject.

108. The method of claim 106, wherein the advertisements are displayed or viewed through a social media channel, social networking, pregnancy education, pregnancy tracker, menstruation tracker, ovulation tracker, or fertility tracker platform.

109. The method of claim 106, wherein recruiting the subject comprises, responsive to the subject responding to the displayed advertisements, directing the subject to visit or view one or more web pages of a user experience (UX) journey.

110. The method of claim 104, wherein the recruiting comprises displaying a contact form to the subject for completing, and receiving the completed contact form comprising personal contact information from the subject.

111. The method of claim 104, wherein the recruiting further comprises displaying an eligibility questionnaire or a set of eligibility criteria to the subject via the one or more web pages.

112. The method of claim 111, further comprising collecting response data from the subject using the eligibility questionnaire, a self-reported family history survey, a demographic survey, a medical condition survey, a customer satisfaction survey, or a combination thereof.

113. The method of claim 112, wherein evaluating the subject for participation in the clinical trial comprises processing the response data collected from the subject.

114. The method of claim 113, wherein processing the response data collected from the subject comprises determining a gestational age of the fetus of the subject and/or a time window for the collection of the biological sample of the subject.

115. The method of claim 96, wherein evaluating the subject for participation in the clinical trial comprises applying an inclusion criterion or an exclusion criterion of the clinical trial.

116. The method of claim 115, further comprising selecting the subject for the clinical trial when the subject satisfies the inclusion criterion.

117. The method of claim 116, further comprising obtaining informed consent from the subject.

118. The method of claim 115, further comprising excluding the subject for the clinical trial when the subject satisfies the exclusion criterion.

119. The method of claim 96, wherein the biological sample is selected from the group consisting of a blood sample, a urine sample, a stool sample, a saliva sample, a vaginal sample, a cervical sample, and a swab sample.

120. The method of claim 119, wherein the biological sample comprises the blood sample.

121. The method of claim 120, wherein the blood sample is obtained from the subject by a phlebotomy service.

122. The method of claim 96, wherein (c) comprises shipping a biological sample collection kit to the location, and receiving a return shipment of the collected biological sample from the location.

123. The method of claim 96, wherein the clinical trial is an interventional clinical trial, an observational clinical trial, or a longitudinal clinical trial.

124. A method for directing a clinical trial, comprising:
(a) evaluating a subject for participation in the clinical trial, wherein the clinical trial is for assessing pregnancy-related states of fetuses of subjects; and
(b) selecting the subject for the clinical trial, based at least in part on the evaluating in (a), wherein the clinical trial comprises collection of data in absence of the subject visiting a health care facility.

125. A system comprising one or more computer processors that are individually or collectively programmed to implement a method for directing a clinical trial, the method comprising:
(a) evaluating a subject for participation in the clinical trial, wherein the clinical trial is for assessing pregnancy-related states of subjects or fetuses thereof;
(b) selecting the subject for the clinical trial, based at least in part on the evaluating in (a); and
(c) directing collection of a biological sample of the subject at a location remote with respect to a health care facility.
